(19) United States
(12) Patent Application Publication
(10) Pub. No.: US 2024/0401075 A1
(43) Pub. Date: Dec. 5, 2024

(54) VECTOR FOR CANCER TREATMENT

(71) Applicant: CANCER RESEARCH TECHNOLOGY LIMITED, London (GB)

(72) Inventors: Lian Ni LEE, Oxford (GB); Senthil CHINNAKANNAN, Oxford (GB); Paul KLENERMAN, Oxford (GB)

(21) Appl. No.: 18/418,855

(22) Filed: Jan. 22, 2024

Related U.S. Application Data
(63) Continuation of application No. 17/721,455, filed on Apr. 15, 2022.
Application No. 17/721,455 is a continuation of application No. PCT/GB2020/052620, filed on Oct. 16, 2020.

(30) Foreign Application Priority Data
Oct. 16, 2019 (GB) 19149848
Jun. 19, 2020 (GB) 20094207

(51) Int. Cl. C12N 15/86; A61K 38/17; A61K 38/47; A61K 39/00; A61K 45/06; A61P 35/00

(52) U.S. Cl. CPC C12N 15/86; A61K 38/1774; A61K 38/47; A61K 39/0011; A61K 39/001188; A61K 45/06; A61P 35/00; C12Y 302/01117; C12N 2710/10343; C12N 2710/10351; C12N 2710/10371

(57) ABSTRACT

The present invention relates to an adenoviral vector or adeno-associated virus vector comprising a nucleotide sequence encoding a single cancer specific CD8+ T cell epitope, wherein the vector is capable of inducing an inflating memory CD8+ T cell response wherein said vector does not comprise a nucleic acid encoding further cancer specific T cell epitopes. It also relates to methods and uses of the vector.

### CROSS-REFERENCE PARAGRAPH

This application is a continuation of U.S. application Ser. No. 17/721,455, filed Apr. 15, 2022, which is a continuation of International Application No. PCT/GB2020/052620 filed Oct. 16, 2020, which claims priority to GB 2009420.7 filed Jun. 19, 2020 and GB 1914984.8 filed Oct. 16, 2019, the disclosures of which are hereby incorporated by reference in their entirety.

### SEQUENCE LISTING

The instant application contains a Sequence Listing which has been submitted electronically in XML file format and is hereby incorporated by reference in its entirety. Said XML copy, created on Jan. 19, 2024, is named 63274_701_302_SL.xml and is 79,551 bytes in size.

### FIELD OF INVENTION

The present invention relates to vectors which are capable of eliciting an inflating memory CD8+ T cell response. These vectors which elicit an inflating memory CD8+ T cell response are suitable for use in the treatment of cancer. The present invention also relates to methods for making the vectors and methods for inducing an inflating memory CD8+ T cell response.

### INTRODUCTION

Anti-cancer strategies aiming to activate the CD8 T cell arm of immunity have shown remarkable efficacy. There is considerable overlap between the requirements for a good CD8 T cell response against a chronic infection with that against cancer—they have to be durable, functional, sustained and able to home to the correct site and resist exhaustion owing to prolonged TCR stimulation.

Epitope based cancer vaccines are one strategy that has been used to activate a T cell response to specific tumour associated antigens. Initially, peptide-based single epitope vaccines were used, however these provided poor clinical responses as they did not adequately active the innate immune system. To enhance the immune activation multi-peptide vaccines were developed, wherein multiple epitopes were administered together.

This approach of administering multiple epitopes has also been performed using adenoviral vectors. By using an adenoviral vector, which has the capacity to encode large transgenes, multiple epitopes can be encoded and delivered as a concatemer (Bei and Scardino., J Biomed Biotechnol 2010:2010:102758). Alternatively, full length antigens can be encoded and delivered. However, there is still a need to improve immune activation against cancer cells.

### SUMMARY OF THE INVENTION

The present invention arises from the surprising finding that a vector encoding a single cancer specific CD8+ and/or CD4+ T cell epitope, referred to herein as a minigene vector, can induce an inflating memory CD8+ T cell response. Memory inflation describes the longitudinal development of stable, expanded CD8+ T cell memory pools, wherein the cells have distinct phenotype and function. This inflating memory response results in a long-lived pool of epitope specific T cells which remain abundant and functional even beyond the acute phase of infection (Klernerman., Immunol Rev 2018 283 (1): 99-11). It is believed that the features of inflating memory cells, may result in an enhanced anti-tumour response.

The present inventors have developed a vaccine platform based on the replication-deficient AdHu5 adenoviral vector backbone in which only the CD8+ T cell epitope of interest is inserted. In this manner the antigen processing requirements are bypassed, which allows inflating responses against otherwise non-inflationary epitopes to develop. It has been demonstrated herein that a single priming injection of the vector resulted in a large epitope specific CD8+ T cell response, wherein the T cells presented inflating memory phenotype. Surprisingly, the responses raised were long-lived, being able to control tumours even >50-90 days after immunization in prophylactic immunization experiments, and when administered into mice already bearing tumours. These responses were detectable for long term, low in PD-1 and also low in checkpoint inhibitors, Lag-3 and Tim-3. In comparison, administration of a vector encoding the full-length protein antigen did not result in a CD8+ T cell response of the same magnitude nor of the same phenotype.

Adenoviral vectors generally provide the advantage of large transgene packaging capacity, due to the removal of one or more viral genes. As such, previous approaches for epitope-based vaccines using adenoviral vectors, have encoded multiple T cell epitopes as a concatemer. However, the present approach has found that a long and durable immune response can be produced by an adenoviral vector comprising a relatively small insert of approx. 70 bp and minimal enhancer elements (referred to herein as a minigene vector). Surprisingly, it has been shown that the short nucleic acid sequence is transcribed in vivo and successfully presented on the MHC molecule, generating peptide specific CD8+ T cells.

Additionally, the magnitude and durability of the CD8+ T cell response generated by the minigene is of a much higher magnitude at the later stages post-delivery (more than 50 days) than previously observed in responses induced using adenoviral vectors containing multiple CD8+ T cell epitopes. By providing an adenovirus or adeno-associated virus encoding a short epitope peptide sequence. it is believed that the encoded peptide circumvents the normal antigen processing requirements for presentation on an MHC molecule. This results in a T cell response which is easier to predict, more reliable, and broader, as well as more robust and effective.

These minigene vectors provide a number of advantages over traditional peptide-based vaccines and DNA vaccines. Firstly, adenoviral vector minigenes are able to induce appropriate priming responses (co-stimulation) within the infected cell. This leads to the generation of potent antigen-specific CD8+ T cell responses. DNA and peptide vaccines are not able to induce priming responses unless combined with an adjuvant. Secondly, adenoviral vector minigenes are able to persistently infect a cell. This characteristic may allow the vector to serve as a long-term source of the antigen, thereby maintaining the size of antigen-specific T cell pool. Thirdly, peptide and DNA vaccines are not able to generate long-lived antigen specific CD8+ T cell responses unless given in multiple prime boost dosing regimens and usually in combination with an adjuvant. By contrast large pools of long-lived antigen-specific CD8+ T cells are generated from a single injection of the minigene. These long-lived tumour specific CD8+ T cell responses are found in the blood and so are present systemically. Therefore, they may play an important role in suppressing micrometastasis after primary tumour control. Finally, the adenoviral vector minigenes also have the advantage of being easy to design and produce, due to the simplicity of the vector and encoded sequence.

As such, the invention relates to an adenoviral vector comprising a nucleotide sequence encoding a single cancer specific CD8+ and/or CD4+ T cell epitope, wherein the vector is capable of inducing an inflating memory CD8+ T cell response.

In an embodiment the invention relates to an adenoviral vector or an adeno-associated virus (AAV) vector comprising a nucleotide sequence encoding a single cancer specific CD8+ T cell epitope, wherein the vector is capable of inducing an inflating memory CD8+ T cell response. In an embodiment the vector is capable of inducing production of CD8+ T cells characterised by markers selected from the group comprising CX3CR1+, KLRG-1+, CD44+, CD62L−. In an embodiment the vector is capable of inducing production of CD8+ T cells characterised by markers selected from the group comprising CX3CR1+, KLRG-1+, CD44+, CD62L−, CD27 (low), CD127 (low). In an embodiment the nucleotide sequence encoding the cancer specific CD8+ or CD4+ T cell epitope comprises from 12 to 45 nucleotide base pairs. In an embodiment the nucleotide sequence encoding the cancer specific CD8+ and/or CD4+ T cell epitope comprises from 24 to 45 nucleotide base pairs. In an embodiment the cancer specific CD8+ and/or CD4+ T cell epitope is derived from a tumour associated antigen. In an embodiment the cancer specific CD8+ and/or CD4+ T cell epitope is mutated in a cancer cell. In an embodiment the cancer specific CD8+ and/or CD4+ T cell epitope is overexpressed in a cancer cell. In an embodiment the cancer specific CD8+ and/or CD4+ T cell epitope is derived from a tumour associated antigen selected from the group consisting of TRP-1, CEA, TAG-72, 9D7, Ep-CAM, EphA3, telomerase, mesothelin, SAP-1 Melan-A/MART-1, tyrosinase, CLPP, cyclin-A1, cyclin-B1 MAGE-A1, MAGE-C1, MAGE-C2, SSX2, XAGE1b/GAGED2a, CD45, glypican-3, IGF2B3, kallikrein-4, KIF20A, lengsin, meloe, MUC5AC, survivin, PRAME, SSX-2, NY-ESO-1/LAGE1, gp70, MC1R, TRP-1/−2, β-catenin, BRCA1/2, CDK4, foetal protein SIM1. In an embodiment the cancer specific CD8+ or CD4+ T cell epitope comprises SEQ ID NO:1 (SPSYVYHQF) or SEQ ID NO:2 (SLLMWITQC). In an embodiment the cancer specific CD8+ and/or CD4+ T cell epitope is specific for colorectal cancer, prostate cancer, oesophageal cancer, liver cancer, renal cancer, lung cancer, breast cancer, breast cancer, pancreatic cancer, brain cancer, hepatocellular cancer, lymphoma, leukaemia, gastric cancer, cervical cancer, ovarian cancer, thyroid cancer, melanoma, carcinoma, head and neck cancer, skin cancer, nasopharyngeal cancer, Epstein Barr driven cancers, Human Papilloma virus driven cancers and soft tissue sarcoma. In an embodiment the vector is human serotype 5 (AdHu5). In an embodiment the vector comprises a CMV promoter. In an embodiment the vector comprises a TATA box. In an embodiment the vector lacks the E1 and E3 proteins. In an embodiment the vector does not comprise any additional nucleotide sequence encoding a cancer specific CD8+ and/or CD4+ T cell epitope. Thus, the vector has a nucleotide sequence encoding a single cancer specific CD8+ T cell epitope and may comprise other vector elements necessary for the transcription of the nucleic acid, but it does not include a nucleic acid sequence that encodes a cancer specific epitope that is not a CD8+ T cell epitope, e.g. a CD4+ T cell epitope. Moreover, it does not include more than one cancer specific CD8+ or CD4+ T cell epitope. Thus, the presence of multiple anti-cancer T cell epitopes in the vector is excluded. This excludes multiple copies of the same anti-cancer T cell epitope or copies of different anti-cancer T cell epitopes. The vector does not have a concatemer, that is a long continuous DNA molecule that contains multiple copies of the same cancer specific T cell epitope linked in series.

In an aspect the invention relates to an immunogenic composition, comprising the vector according to the invention.

In an aspect the invention relates to an immunogenic composition or vaccine composition comprising at least 1, 2, 3, 4, 5, 6, 7, 8, 9 or 10, up to 20, 30, 40 or 50 vectors according to the invention.

In an aspect the invention relates to a host cell, comprising the vector according to the invention, or the immunogenic composition according to the invention.

In an aspect the invention related to the vector or composition according to the invention, for use in therapy.

In an aspect the invention relates to a method of treating or preventing a cancer, comprising administering a therapeutically effective amount of the vector or composition according to the invention.

In an aspect the invention relates to a method of inducing an inflating memory CD8+ T cell response, comprising the step of: administering a therapeutically effective amount of the vector or composition according to the invention, to a subject in need thereof, wherein the CD8+ T cells are characterised by markers selected from the group comprising CX3CR1+, KLRG-1+, CD44+ and CD62L−.

In an aspect the invention relates to a method of producing the vector are described above, comprising the steps of:
i) synthesising the nucleic acid sequence encoding the epitope, as a sense and antisense primer,
ii) cloning the nucleic acid sequence encoding epitope sequence into a first plasmid,
iii) cloning the sequence comprising the nucleic acid sequence encoding epitope into a second plasmid comprising the adenoviral DNA

In an aspect the invention relates to a kit comprising the vector according to the invention, one or more additional active ingredients, pharmaceutically acceptable carrier, diluent, excipient or adjuvant, and optionally instructions for use.

In an aspect the invention relates to a method for inducing a T cell immune response in an animal against a cancer specific CD8+and/or CD4+ T cell epitope, comprising contacting a cell with the vector or composition according to the invention.

### BRIEF DESCRIPTION OF THE DRAWINGS

FIGS. 1A-1E show immunization of Balb/c mice with an AdHu5 replication-deficient vector encoding an AH1 CD8+ T cell tumour epitope stimulates a durable CD8+ T response in the periphery.

FIG. 1A shows schematic representation of the constructs used for the production of AdHu5 vector expressing an MHC-1 binding CT26-specific cancer epitope.

FIG. 1B shows FACs plots showing % CD8+ AH1 tetramer+(tet+) cells in the blood from AH1 (left) and Ad-I8V (right) vaccinated mice.

FIG. 1C shows AHI-tetramer-specific CD8+ T cell responses in the blood at day 7 (left) and day 50 (right) from two independent experiments.

FIG. 1D shows FACS plots showing the presence of indicated markers on CD8+ AH1-tet+ (left) and AH1-tet-(right) populations in the blood from the same sample.

FIG. 1E shows Phenotype of AH1-tet+CD8+ T cells compared to AH1-tet-CD8+ T cells from the same groups at day 7 (left) and day 50) (right) from two independent experiments Geo M FI=geometric mean fluorescence intensity.

FIGS. 2A-2K show memory inflationary AH1-specific T cells demonstrate inhibition of CT26 tumour growth in Balb/c mice after both prophylactic and therapeutic vaccination with Ad-AH1.

FIG. 2A shows Experimental setup for prophylactic vaccination (independently performed twice, P1 and P2) and therapeutic vaccination (T1). A star indicates presence of palpable tumours.

FIG. 2B shows tumor growth curves for the different groups (N=5 per group) in a prophylactic (P1 setting.

FIG. 2C shows tumor growth curves for the different groups (N=5 per group) in a prophylactic (P2) setting.

FIG. 2D shows tumor growth curves for the different groups (N=5 per group) in a therapeutic vaccination setting (T1). In T1, the arrow indicates the time-point of vaccination. Mice vaccinated with Ad-AH1 (1×10⁸ IU) are shown in upright triangle, Ad-A H 1 Low (1×10⁷ IU) in star, Ad-AH1 (1×10⁸ IU)+Ad-GSW11 (1×10⁸ IU) in inverted triangle, Ad-GSW11 (1×10⁸ IU) in circle, Ad-I8V (1×10⁸IU) in grey, and naïve mice in black. TF=tumour free.

FIGS. 2E, 2G, and 2I show statistically significant differences in tumour sizes between groups at day 18 post-challenge. Dots indicate individual mice.

FIGS. 2F, 2H, and 2J depicts graphs showing the slope of the tumour growth curves determined by linear regression from the day tumours show clear tumour growth (day 7 post-challenge for controls and day 18 post-challenge for Ad-AH1 vaccinated mice).

FIG. 2K shows the tumour growth rate was recalculated to determine the specific growth rate. The tumor growth rate between implantation and humane endpoint was quantified using the parameter of specific growth rate (SGR, %/day) calculated using the following equation:

SGR=1n(V2/V1)/(t2−t1), [15]

where V1 and V2 are the tumor volumes at one day post implantation (VI was fixed at 0.01 mm) (t1=Day 0) and endpoint (t2), respectively.

FIGS. 3A-3C show AH1-specific CD8+ T cells differ between tumour and spleen in both abundance and phenotype.

FIG. 3A shows representative FACS plots showing % CD8+ AH1-tet+ cells in the tumour (upper panel) and spleen (lower panel) from Ad-AH1 vaccinated mice. For negative controls, tumour and spleen samples were stained with the full range of fluorochrome-conjugated antibodies and an irrelevant H2-Ld-binding tetramer (pp89) for tumour samples or no tetramer for spleen samples (no tet).

FIG. 3B depicts show % CD8+ AH1-tet+ cells in the tumour (upper panel) and spleen (lower panel) from prophylactic (left panel) and therapeutic (right panel) vaccinated mice.

FIG. 3C shows Heatmap showing phenotype of AH1-specific CD8+ T cells in tumour and spleen from prophylactic vaccinated (Ad-AH1) and control mice (Ad-18V and naïve). Values in cells indicate mean of two independent experiments (N=5−10). Markers quantified by geometric MFI have been normalized to a 0-100% scale.

FIG. 4A shows in tumours from Ad-AH1 vaccinated mice, presence of regulatory T cells (CD4+ FoxP3+ cells) appear to be lower compared to control mice after both prophylactic (left) and therapeutic vaccination (right). Data from Ad-AH1 and Ad-AH1+Ad-GSW11 vaccinated mice was grouped likewise to Ad-18V vaccinated and naïve mice (indicated by vaccinated and controls, respectively).

FIG. 4B shows AdHu5-AH1-MG immunization increases the percentage of Trm tet+ cells in the tumour. Mice immunized with AdHu5-AH1-MG, either singly or in combination, show increased percentages of AH-1 specific CD8 T cells in the tumour (TIL) with a resident-memory phenotype compared to control mice (naive or immunized with irrelevant AdHu5 constructs (AdHu5-18V-MG or AdHu5-GSW11)).

FIGS. 5A-5D show AdHu5-AH1-MG immunization induces AH-1+ CD8 T cells in the spleen that remain functional during tumour growth.

FIG. 5A shows splenocytes and TILs were stimulated with AH-1 peptide to measure their cytotoxic potential based on IFN-gamma secretion.

FIG. 5B shows AH-1-peptide specific splenocytes from immunized animals are able to respond to peptide stimulation.

FIGS. 5C-5D show in contrast, CD8 T cells in the TIL did not respond to peptide stimulation: however the levels of IFN-g secreted in response to PMA-ionomycin was also low indicating a general state of CD8 T cell downregulation in the tumour.

FIG. 6A shows the figures show the correlation between the slope of the tumour growth curve for each animal (indicated with a dot) and its percentage of CD8+ AH-1-specific T cells in the blood (left), spleen (middle) and absolute number of CD8+ AH1 tet+ cells in the tumour (right). The data is shown for two independent prophylactic (P1 and P2) and a single therapeutic (T1) experiment. A lower tumour growth rate correlates with increased levels of AH1-specific CD8+ T cells in spleen and blood post-tumour challenge but weakly correlates with absolute numbers of AH-1 specific CD8 T cells in the tumour.

FIG. 6B shows the comparison of antigen-specific cells from various compartments with the specific growth rates.

FIG. 7A shows therapeutic immunization with an AdHu5 vector encoding full-length gp90) (AdHu5-gp90FL) does not confer similar level of tumour control. Specific growth rate of tumors in each group was compared using Mann Whitney tests. *p<0.05, ** p<0.005.

FIG. 7B shows mice who cleared the tumour by therapeutic and prophylactic immunization continue to bear AH-1 specific cells in circulation. The blood of mice who completely cleared tumours 6 month previously were sampled and stained for AH1+CD8 T cells by tetramer staining.

FIGS. 8A-8G show HHD mice immunized with AdHu5− NY-ESO-1₁₅₇₋₁₆₅ minigene construct develop a long-lived circulating population of NY-ESO-1₁₅₇₋₁₆₅ Tet+ CD8 T cells with an inflating memory phenotype. The results shown are from 4-5 mice per group from 1 of 2 independent experiments.

FIG. 8A shows levels of NY-ESO-1157-165 Tet+ cells were measured by tetramer staining in blood after groups of HHD mice (N=4-5 per group) were immunized with 1=110⁸ IU AdHu5-NY-ESO-1 mini or 1×10⁹ I.U. AdHu5-NY-ESO-1− FL. The schematic representation of the constructs used is shown.

FIG. 8B shows the cells of FIG. 8A phenotyped by surface staining with CD44 and CD62L.

FIG. 8C shows memory subset markers of inflating cells CX3CR1 from FIG. 8A.

FIG. 8D shows memory subset marker of inflating cells from FIG. 8A KLRG-1.

FIG. 8E shows markers of exhaustion, PD-1.

FIG. 8F shows marker of exhaustion Tim3.

FIG. 8G shows marker of exhaustion Lag-3.

FIGS. 9A-9D show Mice primed with a single dose of AdHu5-NY-ESO-1157-165 minigene develop higher percentages of circulating NY-ESO-1₁₅₇₋₁₆₅ Tet+CD8 T cells after tumour challenge and exhibit better control of tumour growth. Statistical measurement was performed by T-tests. The data shown are from two separate independent experiments.

FIG. 9A shows At 53 (solid line) or 99 (dashed line) days post-immunization with either 1×110⁸ IU AdHu5-NY-ESO-1 mini or 1×10⁹ I.U. AdHu5-NY-ESO-1-FL, animals were injected subcutaneously (s.c) with either 1×10⁶ (solid line)) or 5×10⁵ (dashed line) HHD-NY-ESO-1 sarcoma cells As negative controls, groups of mice were either immunized with 1×10⁸ I.U of an irrelevant AdHu5-minigene construct (N=5) or left naïve (N=10). The tumours were measured every 1-2 days using digital callipers.

FIG. 9B. Circulating levels of NY-ESO-1₁₅₇₋₁₆₅ Tet+ cells were measured by tetramer staining in blood taken 14 days after tumour challenge.

FIG. 9C The levels of NY-ESO-1₁₅₇₋₁₆₅ Tet+ detected in blood before tumour challenge versus the size of the tumours measured at early (Day 14) are shown.

FIG. 9D The levels of NY-ESO-1₁₅₇₋₁₆₅ Tet+ detected in blood before tumour challenge versus the size of the tumours measured at late, Day 27/28 are shown.

FIGS. 10A-10F show NY-ESO-1₁₅₇₋₁₆₅ Tet+ CD8 T cells from tumours (TIL) display elevated levels of markers of exhaustion and activation. Mice were sacrificed, spleens and tumours were removed and analysed when the humane endpoint was reached, either by unhealed ulceration or when they approached 1300 mm³ in size.

FIG. 10A. Lymphocytes were isolated from both compartments and shows the percentage of CD8 T cells measured.

FIG. 10B shows the percentage of CD8 T+ Tet+ cells measured.

FIGS. 10C-F shows the percentages of NY-ESO-1₁₅₇₋₁₆₅ Tet+ cells in spleen (Spl) and TIL were determined as were levels of the exhaustion markers (FIG. 10C) PD-1. (FIG. 10D) Tim-3 and (FIG. 10E) Lag-3 along with apoptotic marker (FIG. 10F) FasL.

FIGS. 11A-11E show CX3CR1 is preferentially upregulated on NY-ESO-1₁₅₇₋₁₆₅ Tet+ CD8 T cells in spleen and TIL after AdHu5-NY-ESO-1₁₅₇₋₁₆₅ minigene immunization. Lymphocytes isolated from TIL or spleen when the humane endpoint was reached were stained with the tetramer and the levels of the following molecules on Tet+ cells were determined.

FIGS. 11A-11D. The inflating marker CX3CR1 on (FIG. 11A) spleen and (FIG. 11B) TIL. (FIG. 11C) Markers of an effector memory phenotype, CD44 and CD62L and (FIG. 11D) resident memory markers CD103 and CD69.

FIG. 11E shows the levels of CD4+ regulatory T cells (Treg) in both compartments were also determined by intracellular staining.

FIGS. 12A-12F show CX3CR1+ CD8 T cells are more resistant to oxidative stress and contain higher levels of healthy polarized mitochondria.

FIG. 12A shows the levels of intracellular reactive oxygen species (ROS) in CX3CR1+/-gfp splenocytes from Ad-lacZ or MCMV infected mice at day >50 post-infection were detected by CellROX Red assay. (N=2 independent experiments).

FIG. 12B shows peripheral blood lymphocytes from C57BL/6 mice infected >100 days previously with MCMV or an AdHu5 recombinant adenovector (Ad-I8V) were stained with MitoTracker Green (detects all mitochondria) and MitoTracker DeepRed (detects only healthy. polarized mitochondria), then surface stained with anti-mouse CD8, anti-mouse CX3CR1, LiveDead nearIR Fixable Marker and then analysed on an LSRII and the data calculated on FlowJo.

FIG. 12C shows antigen-specific CX3CR1+ inflating cells contain healthier mitochondria and show enhanced redox resilience.

FIGS. 12D-12E show that when incubated in serum-free media (i.e., stress), there was a marked survival of the CX3CR1+ population compared to CX3CR1 negative T cells (FIG. 12D) in the bulk and antigen-specific populations (FIG. 12E).

FIG. 12F shows the levels of reactive oxygen species (ROS) upon serum starvation indicating that CX3CR1+ T cells (bulk and antigen-specific) possess intrinsically lower levels of reactive oxygen species and are more resistant to oxidative stress.

FIG. 13 shows preventative immunization with HPV16 E7⁴⁹⁻⁵⁷ minigene vector confers protection against tumour challenge. E7⁴⁹⁻⁵⁷ specific cells are able to traffic to site of tumour implantation and confer protection against tumour challenge.

FIG. 14. Shows synergistic effect after immunization with a panel minigenes encoding CD8 T cell epitopes against MCMV at a suboptimal dose. A panel of 3 minigenes against known MCMV-specific CD8 T cell epitopes, namely M45 (⁹⁸⁵HGIRNASFI⁹⁹³), M38 (³¹⁶SSPPMFRV³²⁵) and m139 (⁴¹⁹TWYGFCLL⁴²⁶) were constructed. These were injected i.v. into C57BL/6 mice either as individual minigenes or as a cocktail. The minigene encoding M38 and M139 were injected at a suboptimal dose of 1×10⁷ infectious units (I.U) while the minigene encoding M45 was injected at the optimal dose of 1×10⁸ I.U. The levels of M38-specific cells in the blood at Day 6 post-immunization was measured. Surprisingly, mice that received the combination minigene vaccine containing M38-minigene and m139-minigene vectors at suboptimal doses, plus M45-minigene at optimal dose, developed higher levels of M38-specific T cells compared to the groups injected with only a sub-optimal dose of M38-minigene vector alone. This unexpected result suggests that delivery of a cocktail of minigene vectors at suboptimal doses may have additive effect to enhance the magnitude of the antigen-specific T cell over that observed in upon immunization with the single vector alone.

FIGS. 15A-15B show CD8 T cells from the tumours of AdHu5-AH1-MG immunized mice express higher levels of granzyme-B.

FIG. 15A shows levels of granzyme B in total CD8 T cells in the tumours 23 days post-implantation, 16 days post immunization and tumour sizes at time of analysis.

FIG. 15B shows levels of the transcription factors T-bet and Eomes in AH1-specific CD8 T cells in the tumours 23 days post-implantation, 16 days post immunization.

FIGS. 16A-16D show testing the GP70₄₂₃₋₄₃₁ (AH1) minigene as a therapeutic vaccine in combination with anti-PD-L1.

FIG. 16A shows groups of mice immunized with the indicated adenovectors 7 days after tumour challenge were then treated with anti-PD-L1 or isotype control. The tumour sizes of the individual mice are shown.

FIG. 16B shows survival curve of all groups of mice.

FIG. 16C shows the % of GP70₄₂₃₋₄₃₁ (AH1) Tet+ cells in circulation 15 days after immunization (22 days post-tumour challenge).

FIG. 16D shows the specific growth rate of tumors in each group was compared using Mann-Whitney test. *p<0.05, **p<0.005

FIG. 17A, FIG. 17B, FIG. 17C, and FIG. 17D show Spleen- and tumour-derived single cells from prophylactic (FIG. 17A, FIG. 17C), or therapeutic (FIG. 17B, FIG. 17D) therapeutic vaccination were stimulated ex vivo with AH1-peptide (4 μg/ml) or PMA-Ionomycin (IO) for 7 hours and then stained for intracellular cytokine production of IFNγ. For each sample, low-level background activation (media only) was subtracted.

FIGS. 17E-17H shows spleen and tumour-derived single cells from therapeutic vaccination combined with anti-PD-L1 were stimulated ex vivo with AH1-peptide (4 μg/ml) or PMA-Ionomycin (IO) for 7 hours and then stained for intracellular cytokine production of IFNγ. For each sample, low-level background activation (media only) was subtracted. The CD8 T cell response in spleen (FIG. 17E) and tumour (FIG. 17G) and CD4 T cell response in spleen (FIG. 17F) and tumour (FIG. 17H) are shown.

FIGS. 18A-18F show Pilot experiment to determine if two minigenes encoding two tumour antigens will improve tumour control. A shows protocol used—tumour implantation performed on day 0, vaccination with one of AdHu5-AH1 minigene (MG), AdHu5-e2F8-27mer MG, Combo (both MG—AdHu5-AH1 and AdHu5-e2F8-27mer), irrelevant AdHu5-MG, unvaccinated on day 7, N=6 per treatment group. Half of each group was treated with the checkpoint inhibitor anti-PD-1 and half the group were treated with an isotype control at 12, 16 and 19 days post-implantation. Bleeds were performed on days 13 and 20 (FIG. 18A).

FIGS. 18B-18F show the tumour growth over time.

FIGS. 19A-19B show comparison of the combination minigene treatment plus ant-PD-1 compared to negative controls and vaccination with a single minigene.

FIG. 20, Growth rates of tumours calculated by linear regression for the combination minigene treatment, single minigene treatment and negative control.

FIG. 21, % CD8+ AH1-tet+ cells and % CD8+ ef28-tet+ cells produced from vaccination with the combination minigene treatment and vaccination with the single minigenes AdHu5-AH1 and AdHu5-e2F8-27mer measured 6 days post-vaccination.

FIG. 22 shows Simultaneous i.v. immunization with two minigene constructs/vaccines (combo—AdHu5-AH1 and AdHu5-e2F8) induces both antigen-specific populations at similar magnitudes and phenotype to single vaccine measured 11 days post-vaccination.

FIG. 23. Shows immunization with two minigenes targeting CD8 T cell epitopes (AdHu5-AH1 and AdHu5-e2F8) in a cancer cell controls tumor growth. The linear regression data in FIG. 20 has been recalculated as specific growth rate.

FIGS. 24A-24B show Transcriptional profiling of an unconventional subset of memory T cells: inflating memory T cells. (FIG. 24A) A PCA of Inflating/non-Inflating CD8 T cells. 3D PCA showing distribution of transcription profiles of two independent models of Inflating samples (M38, D8V—later stages i.e. inflating memory, circled in blue) and non-Inflating Samples (M45, I8V—later stages, i.e. central memory, circled in brown), at acute stages (days 7 or 21) and later stages (days 50 or 100), and naive samples. (FIG. 24B) PCA of Exhausted/non-Exhausted CD8 T cells. 3D PCA showing distribution of transcription profiles of a model of Exhaustion (Cl13, Tetrahedrons—day 30 are circled in grey), with non-Exhaustive samples (Arm, spheres—day 30 are circled in blue) at different stages, and naive samples. Stages: 6 days (yellow), 8 days (brown), 15 days (pink), 30 days (black), naive (green).

FIG. 25A-25C show the inflating memory subset express a distinct gene module compared to other T cell memory subsets. (FIG. 25A) Weighted Gene Co-expression Network Analysis of Inflating samples. Gene co-expression network analysis detected 6 gene modules (merging distance=0.25, soft-thresholding power β=9); Blue module (highlighted) genes are enriched with immune relevant GO categories and contains relevant genes such as Tbx21, Eomes, Zeb2, and E2f2. (FIG. 25B) PCA of Inflating/Exhausted samples based on Blue module genes. PCA plot using the first three principal components and based on a gene set of 588 genes, detected as blue module in Gene co-expression network analysis of Inflating samples only. The plot shows distribution of Naïve (green). Non-inflating and Non-exhausting (blue), and Inflating and Exhausting (red) samples (spheres: Exhaustion study: tetrahedron: Inflation study) (The inflating memory population are red tetrahedrons circled in blue). (FIG. 25C) Hierarchical clustering of Inflating/Exhausted samples based on Blue module genes. Dendogram plot showing sample clustering analysis (Euclidian distance) on Inflating-Exhausted merged sets, based on a gene set of 469 genes, detected as blue module in a repeated Gene co-expression network analysis of Inflating samples after removing outliers (Soft-thresholding power β=20). The memory inflation cluster is contained in the rectangle.

FIG. 26A shows the schematic of the AdHu5 adenovirus with minigene immunogen cassette and a close-up view of the minigene immunogen cassette. FIG. 26B shows the schematic of the AAV ITR with minigene immunogen cassette and a close-up view of the minigene immunogen cassette.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous. The practice of the present invention will employ, unless otherwise indicated, conventional techniques of immunology, molecular biology, chemistry, biochemistry and recombinant DNA technology, which are within the skill of the art. Such techniques are explained fully in the literature, see, e.g., Green and Sambrook et al., Molecular Cloning: A Laboratory Manual, 4th ed., Cold Spring Harbor Laboratory Press, Cold Spring Harbor, N.Y. (2012).

The present invention is based on the surprising finding that an adenoviral vector encoding a single cancer specific epitope results in an inflating memory CD8+ T cell response. The term inflating memory response refers to a sustained, functional, durable CD8+ T cell response. The resulting pool of CD8+ T cells are able to resist exhaustion which can occur due to prolonged TCR stimulation. T cell exhaustion can be characterised by upregulation of markers such as PD-1, Tim-3 and Lag-3.

The inflating memory CD8+ T cells are characterised by a unique phenotype compared to other CD8+ memory subsets, including the expression of markers CX3CR1 and KLRG-1. The cells also demonstrate a distinct transcriptional profile from both central memory and exhausted memory T cell subsets. The cells also demonstrate features such as enhanced redox resilience which may be due to intrinsically lower levels of reactive oxygen species and resilience to oxidative stress. In particular, the transcriptional profile is driven by the transcription factor Tbx21 with minimal contribution from Eomes. This results in a CD8+ T cell phenotype that is long lived, and present in the peripheral organs in high numbers whilst retaining effector function. The antigen-specific inflating memory CD8+ T cells develop through a unique set of processing, presentation and co-stimulation conditions. The processing of the epitope occurs independently of the immunoproteasome and presentation by a non-haematopoietic unconventional APC during the later stages may help to preserve this phenotype. Without wishing to be bound by theory it is thought that by using a vector of the present invention, which encodes a single epitope of interest, the antigen processing requirements are bypassed thereby resulting in an inflating memory response.

In an embodiment the invention relates to an adenoviral vector comprising a nucleotide sequence encoding a single cancer specific CD8+ and/or CD4+ T cell epitope, wherein the vector is capable of inducing an inflating memory CD8+ T cell response. In an embodiment the adenoviral vector comprises a nucleotide sequence encoding a single cancer specific CD8+ and/or CD4+ T cell epitope, e.g. a single cancer specific CD8+ T cell epitope, and the vector does not comprise any additional cancer specific CD8+ and/or CD4+ T cell epitopes. As such the vector of the present invention encodes a single cancer specific CD8+ and/or CD4+ T cell epitope, e.g. a single cancer specific CD8+ T cell epitope. The present invention does not extend to adenoviral vectors encoding more than one or multiple cancer specific CD8+ and/or CD4+ T cell epitopes.

In an embodiment the invention relates to an adenoviral vector comprising a nucleotide sequence encoding a single cancer specific CD8+ T cell epitope, wherein the vector is capable of inducing an inflating memory CD8+ T cell response. In an embodiment the adenoviral vector comprises a nucleotide sequence encoding a single cancer specific CD8+ T cell epitope, and the vector does not comprise any additional cancer specific CD8+ T cell epitopes. As such the adenoviral vector of the present invention encodes a single cancer specific CD8+ T cell epitope. The present invention does not extend to adenoviral vectors encoding more than one or multiple cancer specific CD8+ T cell epitopes.

The vector of the present invention, which encodes a single cancer specific CD8+ T cell epitope, is able to generate a sustained, functional, durable CD8+ T cell response from a single dose. The resulting pool of CD8+ T cells are able to resist exhaustion which can occur due to prolonged TCR stimulation. The resulting pool of CD8+ T cells may also demonstrate enhanced redox resilience and low levels of reactive oxygen species.

As used herein the term “vector” refers to a nucleic acid sequence capable of transporting into a cell another nucleic acid to which the vector sequence has been linked. The vectors of the present invention are adenoviral and comprises the nucleotide sequence encoding a single cancer specific CD8+ or CD4+ T cell epitope containing a gene construct in a form suitable for expression by a cell (e.g., linked to a transcriptional control element).

As used herein the term “epitope” refers to a part of an antigen that is recognised by the immune system which may be a short protein sequence. A “cancer specific CD8+ and/or CD4+ T cell epitope” refers to an epitope that may be presented by an antigen presenting cell bound to an MHC molecule which are then recognised by the T-cell receptor (TCR). CD4+ T cells express the CD4 coreceptor, which binds to MHC II, and recognize peptides presented by MHC II molecules. CD8+ T cells express the CD8 coreceptor, which binds to MHC I, and recognize peptides presented by MHC I molecules.

Inflating memory T cells can be characterized by the presences of specific markers and cell surface markers. Methods to identify and quantify these markers are well known in the art. Examples of suitable methods include but are not limited to affinity-based separation methods, magnetic cell sorting techniques, fluorescence-based cell sorting techniques such as FACS (fluorescence activated cell sorting). The inflating memory CD8+ T cells can be characterised by the presence of a number of markers, examples include but are not limited to CX3CR1, KLRG-1, CD44. The inflating memory CD8+ T cells can also be characterised by the low expression of a number of markers, example include but are not limited to CD62L, CD27, CD127. The term “low expression” may refer to cells wherein there is no expression of the markers, it may also refer to cells wherein there is low expression of the markers relative to other cells in the sample.

In an embodiment the inflating memory CD8+ T cells are characterised by markers selected from the group comprising CX3CR1+, KLRG-1+, CD44+, CD62L−, wherein the designation (+) indicates the presence of the marker, and the designation (-) indicates low expression or no expression of the marker. Wherein the (−-) designation means low expression this may be further indicated by “(low)”. The inflating memory CD8+ T cells may be characterised by markers selected from the group comprising CX3CR1+, KLRG-1+, CD44+, CD62L−, CD27−(low), CD127−(low).

The inflating memory CD8+ T cells may be characterised by the phenotype CX3CR1+, KLRG-1+, CD44+, CD62L−. The inflating memory CD8+ T cells may be characterised by the phenotype CX3CR1+, KLRG-1+, CD44+, CD62Lm −, CD27−(low), CD127−(low).

The CD8+ T cells produced in an inflating memory response may have a number of other characteristics. For example, the cells comprise a transcriptional profile driven by Tbx21 (also referred to as T-bet). These cells show a sustained expression of Tbx21. The cells may also show a sustained expression of E2f2 a transcription factor generally involved in cell growth and proliferation. The cells may also lack expression or have low expression of the transcription factor Eomes.

The inflating memory CD8+ T cells may not demonstrate classical contraction after exposure to an antigen. During classical memory evolution after exposure to an antigen the cells form a contracted central memory pool which makes up <1% of total circulating CD8+ T cells. However, inflating memory cells are maintained as large pools of cells which circulate in the blood. As such, in an embodiment the resulting inflating memory CD8+ T cells form approximately 2% to approximately 20% of total CD8+ T cells, preferably approximately 8% to approximately 20% of total CD8+ T cells, more preferably approximately 12% to approximately 20% of total CD8+ T cells.

In an embodiment the large pools of inflating memory CD8+ T cells retain their effector memory phenotype. The resulting inflating memory CD8+ T cells may retain their memory effector phenotype for a prolonged period, wherein the effector phenotype is characterised by CD44+, CD62L−. The inflating memory CD8+ T cells may retain their memory effector phenotype for up to 60 days post exposure to the vector of the present invention, up to 55 days post exposure to the vector of the present invention, up to 50 days post exposure to the vector of the present invention, up to 40 days post exposure to the vector of the present invention, or up to 30 days post exposure to the vector of the present invention.

The inflating memory CD8+ T cells may also lack markers of exhaustion. T cell exhaustion can occur from excessive TCR (T cell receptor) stimulation. Markers of T cell exhaustion can include upregulation of markers such as PD-1, Tim-3, Lag-3. As such, in an embodiment the inflating memory CD8+ T cells may lack or demonstrate low expression of markers selected from the group consisting of PD-1, Tim-3, Lag-3.

The nucleotide sequence encoding a single cancer specific CD8+ and/or CD4+ T cell epitope may comprise from approximately 12 to approximately 45 base pairs. in another embodiment the nucleotide sequence may comprise approximately 15 to approximately 45 base pairs, in another embodiment the nucleotide sequence may comprise approximately 18 to approximately 45 base pairs, in another embodiment the nucleotide sequence may comprise approximately 21 to approximately 45 base pairs, in a preferred embodiment the nucleotide sequence may comprise approximately 24 to approximately 45 base pairs. As such, the vector encodes a single cancer specific CD8+ and/or CD4+ T cell epitope comprising approximately 5 to approximately 15 amino acids, in another embodiment the vector encodes an epitope comprising approximately 6to approximately 15 amino acids, in another embodiment the vector encodes an epitope comprising approximately 7 to approximately 15 amino acids, in a preferred embodiment the vector encodes an epitope comprising approximately 8 to approximately 15 amino acids.

The single cancer specific CD8+ and/or CD4+ T cell epitope is an immunogenic epitope, in that it elicits an immune response. T cell epitopes bind to the major histocompatibility complex in order to initiate a subsequent immune response. As such in an embodiment the epitope is capable of binding and presenting on an MHC molecule. There are multiple methods known in the art to identify epitopes which bind the MHC and therefore produce an immune response. These methods include peptide-MHC binding prediction models of which there are multiple programs publicly available.

In an embodiment the single cancer specific CD8+0 and/or CD4+ T cell epitope is derived from a tumour associated antigen (TAA). A TAA is an antigenic product produced by a cancer and it provides a biomarker for targeted identification of a tumour. TAAs can be broadly categorized into aberrantly expressed self-antigens, mutated self-antigens and tumour specific antigens. As such, the TAA may be upregulated or over-expressed in the cancer cell. The TAA may be mutated within the cancer cell. The TAA may be specific for the cancer cell and only expressed within the cancer cell, this may also be referred to as a tumour specific antigen.

In an embodiment the cancer specific CD8+ and/or CD4+ T cell epitope is mutated in a cancer cell. In an embodiment the cancer specific CD8+ and/or CD4+ T cell epitope is overexpressed in a cancer cell. In an embodiment the cancer specific CD8+ and/or CD4+ T cell epitope is a non-coding tumour specific epitope. As used herein the term “non-coding tumour specific epitope” refers to a peptide found on a cancer cell, wherein the peptide is derived from a nucleotide sequence that is epigenetically suppressed in healthy cells. These peptide sequences are aberrantly expressed within tumour cells.

In an embodiment the cancer specific CD8+ and/or CD4+ T cell epitope is not a cryptic epitope. As used herein a “cryptic epitope” refers to refers to an epitope which is not immunogenic in immunocompetent individuals.

In an embodiment the cancer specific CD8+ and/or CD4+ T cell epitope may be a viral epitope that is associated with a virally driven cancer. The virally driven cancer may be HPV (human papilloma virus), HTLV (human T-lymphotropic virus), or EBV (Epstein Barr virus).

In an embodiment the cancer specific CD8+ and/or CD4+ T cell epitope is derived from a tumour associated antigen selected from the group consisting of TRP-1, CEA, TAG-72, 9D7, Ep-CAM, EphA3, telomerase, mesothelin, SAP-1 Melan-A/MART-1, tyrosinase, CLPP, cyclin-A1, cyclin-B1 MAGE-A1, MAGE-C1, MAGE-C2, SSX2, XAGE1b/GAGED2a, CD45, glypican-3, IGF2B3, kallikrein-4, KIF20A, lengsin, meloe, MUC5AC, survivin, PRAME, SSX-2, NY-ESO-1/LAGE1, gp70, MC1R, TRP-1/-2, β-catenin, BRCA1/2, CDK4.

The cancer specific CD8+ and/or CD4+ T cell epitope may be a private epitope. As used herein the term “private epitope” refers to an epitope which is found exclusively on a single antigen in the cancer of a single person. The cancer specific CD8+ and/or CD4+ T cell epitope may be a public epitope. As used herein the term “public epitope” refers to an epitope that is found on the cancer of two or more people.

In an embodiment the cancer specific CD8+ and/or CD4+ T cell epitope may be a neoepitope. As used herein the term “neoepitope” refers to epitopes which have arisen through mutations within the tumour cells, in particular somatic or passenger mutations may lead to the production of a neoepitope. In an embodiment the cancer specific CD8+ and/or CD4+ T cell epitope is not a neoepitope.

In an embodiment the cancer specific CD8+ and/or CD4+ T cell epitope is specific for colorectal cancer, prostate cancer, oesophageal cancer, liver cancer, renal cancer, lung cancer, breast cancer, breast cancer, pancreatic cancer, brain cancer, hepatocellular cancer, lymphoma, leukaemia, gastric cancer, cervical cancer, ovarian cancer, thyroid cancer, melanoma, carcinoma, head and neck cancer, skin cancer, nasopharyngeal cancer, Epstein Barr driven cancers, Human Papilloma virus driven cancers and soft tissue sarcoma. The term “cancer” as used herein refers to diseases with abnormal cell growth, as used herein the term refers to both a primary tumour and metastasis of the primary tumour.

In an embodiment the cancer specific CD8+ and/or CD4+ T cell epitope comprises SEQ ID NO:1 (SPSYVYHQF) or SEQ ID NO:2 (SLLMWITQC) or SEQ ID NO:37 (SLLMWITQV). Where the cancer specific CD8+ and/or CD4+ T cell epitope is a viral epitope that is associated with a virally driven cancer the epitope may comprise SEQ ID NO:7 (RAHYNIVTF). The virally driven cancer may be selected from EBV driven cancers, HTL driven cancers, and HPV driven cancers. EBV driven cancers may include Hodgkin Lymphoma (HL), Burkitt Lymphoma (BL), Diffuse Large B cell Lymphoma (DLBCL) and two rarer tumors associated with profound immune impairment, plasmablastic lymphoma (PBL) and primary effusion lymphoma (PEL), LPDs and malignant lymphomas of T or NK cells, nasopharyngeal carcinoma (NPC) and gastric carcinoma of epithelial origin, and leiomyosarcoma. HPV driven cancers may include anogenital cancers, oropharyngeal cancers, oral cavity cancer, head and neck squamous cell carcinoma and laryngeal cancer.

In an embodiment the cancer specific CD8+ and/or CD4+ T cell epitope comprises one or more of the epitopes in Table 1.

TABLE 1

| HLA | | Amino acid | | | |
|---|---|---|---|---|---|
| type | Target-epitope | sequence | SEQ ID NO | Gene | Target cancer |
| Human | | | | | |
| A*0201 | NY-ESO-1₁₅₇₋₁₆₅ | SLLMWITQC | SEQ ID NO: 2 | CTAG1B | Cancers |
| | | | | | expressing NY- |
| | | | | | ESO-1 |
| Mouse | | | | | |
| H-2Ld | MuLV env | SPSYVYHQF | SEQ ID NO: 1 | env gp70 | CT26 |
| | gp70₄₂₃₋₄₃₁ | | | | |
| H-2Dd | MuLV gp90₁₄₇₋₁₄₈ | GGPESFYCA | SEQ ID NO: 3 | env gp70 | CT26 |
| | | SW | | | |
| H-2Kd | E2f8₅₀₉₋₅₃₅ | VILPQAPSGP | SEQ ID NO: 4 | e2f8 | CT26 |
| | | SYATYLQPA | | | |
| | | QAQMLTPP | | | |
| H-Kd | MtCh1₃₆₁₋₃₇₀ | KYLSVQSQLF | SEQ ID NO: 5 | mtch1 | CT26 |
| H-2Kd | Mtch1₃₆₁₋₃₆₉ | KYLSVQSQL | SEQ ID NO: 6 | mtch1 | CT26 |
| and H- | | | | | |
| 2Ld | | | | | |
| H-2Db | HPV16 E7₄₉₋₅₇ | RAHYNIVTF | SEQ ID NO: 7 | | Human |
| | | | | | papillomavirus- |
| | | | | | driven cancers |

Further, cancer specific CD8+ and/or CD4+ T cell epitopes may be determined using techniques know in the art such as proteomics approaches, mass spectrometry approaches, genomic approaches, transcriptome analysis, bioinformatics approaches and in silico methods. It would be possible for the skilled person to select an appropriate epitope to be encoded within the vector of the present invention.

The nucleic acid encoding the cancer specific CD8+ and/or CD4+ T cell epitope may be codon optimised for mammalian codon usage. Suitably the nucleic acid sequence may be codon optimised for human codon usage.

The vector may comprise adeno-associated virus (AAV). The vector may comprise adenovirus.

The adenoviral vector or AAV vector may also have additional features such as enhancer and promoter regions. In an embodiment the vector may comprise a strong promoter examples include but are not limited to a CMV promoter, an RSV promoter, an EF1α promoter. In a preferred embodiment the vector comprises a CMV promoter, a suitable sequence for a CMV promotor is provided in SEQ ID NO:18. In an embodiment the vector may comprise a TATA box. In an embodiment the vector comprises a translation initiation sequence, for example a Kozak sequence. A Kozak sequence has the consensus sequence (gcc)gccRccAUGG, a suitable Kozak sequence is provided in SEQ ID NO:19. In an embodiment the vector comprises a termination sequence and/or a polyadenylation sequence. A suitable polyadenylation sequence is provided in SEQ ID NO:34. The AAV vector may comprise inverted terminal repeat (ITR) sequences. A suitable ITR sequence is provided in SEQ ID NO:42.

In an embodiment the vector does not comprise additional cancer specific CD8+ and/or CD4+ T cell epitopes. The vector only encodes a single cancer specific CD8+ and/or CD4+ T cell epitope. In an embodiment the adenoviral vector consists of the vector back bone, a promoter region and a nucleotide sequence encoding a single cancer specific CD8+ T cell epitope. The adenoviral backbone may comprise additional features such as enhancer regions, promoter regions, TATA box, translation initiation sequence.

The AAV vector may be serotype 1, 2, 3, 4, 5, 6, 7, 8 or 9. In a preferred embodiment the AAV vector may be serotype 2 or 5. The AAV vector may comprise ITR sequences, in a preferred embodiment the ITR sequences flank the encoded cancer specific CD8+ and/or CD4+ T cell epitope. There may be an ITR sequence present 5′ to the cancer specific epitope and an ITR sequence 3′ to the cancer specific epitope. The 5′ ITR sequence may comprise SEQ ID NO:39. The 3′ ITR sequence may comprise SEQ ID NO:42. The AAV vector may comprise sequences 5′ to the cancer specific epitope for example SEQ ID NO:38. The AAV vector may comprise sequences 3′ to the cancer specific epitope for example SEQ ID NO:41. In order to produce the AAV vector comprising the cancer specific CD8+ and/or CD4+ T cell epitope, helper plasmids may be used. A helper plasmid or plasmids may be used to provide genes required for AAV replication or packaging. In an embodiment helper plasmid encodes E2A, E4 and VA adenoviral proteins and or encodes the rep and cap genes of AAV.

The adenoviral vector may be a Species C serotype. Species C includes Ad1, 2, 5 and 6 serotypes. In a preferred embodiment the adenoviral vector is a human serotype 5 (AdHu5). It may be preferable for the adenoviral vector to be modified for example to reduce the immunogenicity and improve biosafety of the vector. As such, the adenoviral vector may be replication-incompetent. The adenoviral vector may lack the E1 and E3 proteins. The adenoviral vector may comprise sequences 5′ to the cancer specific epitope for example SEQ ID NO:13. The adenoviral vector may comprise sequences 3′ to the cancer specific epitope for example SEQ ID NO:14.

Other adenoviral vectors may also be suitable for the vector for the present invention. In an embodiment the vector may be an animal derived adenoviral vector for example canine, simian in particular rhesus monkey and chimpanzee. In an embodiment the adenoviral vector may be a rare serotype vector derived from a non-human primate. Vectors derived from chimpanzee may be suitable for the vector for the present invention, examples include but are not limited to ChAd63, ChAd3, ChAdY25.

In an embodiment there is provided an immunogenic composition, comprising the vector as defined above. The immunogenic composition may further comprise one or more additional active ingredients, pharmaceutically acceptable carrier, diluent, excipient or adjuvant.

The immunogenic composition comprising a vector according to the invention may be used in combination with at least one other immunogenic composition comprising a vector according to the invention, wherein each vector encodes a different cancer specific CD8+ and/or CD4+ T cell epitope. The immunogenic composition comprising a first vector according to the invention may be administered separately, sequentially or simultaneously with an immunogenic composition comprising a second vector according to the invention.

In an embodiment the immunogenic composition may comprise at least two vectors according to the invention. It may be preferable for the at least two vectors to encode different cancer specific CD8+ and/or CD4+ T cell epitopes. Wherein further additional vectors are present in the composition the vector may encode different cancer specific CD8+ and/or CD4+ T cell epitopes. The immunogenic composition may further comprise one or more additional active ingredients, pharmaceutically acceptable carrier, diluent, excipient or adjuvant. Without wishing to be bound by theory, use of a cocktail of vectors encoding different epitopes may result in a stronger immune response, further there may be a synergistic effect which enhances the immune response.

Wherein the composition of the present invention comprises at least two vectors as described herein, the vectors may be provided as separate medicaments for administration at the same time or at different times.

In an embodiment, wherein the composition comprises at least two vectors as described herein, the vectors may be provided as separate medicaments for administration at different times. When administered separately and at different times, either vector may be administered first. In some embodiments, both can be administered on the same day or on different days, and they can be administered using the same schedule or at different schedules during the treatment cycle.

Alternatively, wherein the composition comprises at least two vectors as described herein, the administration of the vectors may be performed simultaneously. Wherein simultaneous administration is used the vectors may be formulated as separate pharmaceutical compositions. In a preferred embodiment the at least two vectors may be formulated as a single pharmaceutical composition.

The composition of the invention can be in the form of a liquid, e.g., a solution, emulsion or suspension. The liquid compositions of the invention, whether they are solutions, suspensions or other like form, can also include one or more of the following: sterile diluents such as water, saline solution, preferably physiological saline, Ringer's solution, isotonic sodium chloride, fixed oils such as synthetic mono or digylcerides, polyethylene glycols, glycerin, or other solvents: antibacterial agents such as benzyl alcohol or methyl paraben: and agents for the adjustment of tonicity such as sodium chloride or dextrose. The composition can be enclosed in an ampoule, a disposable syringe or a multiple-dose vial made of glass, plastic or other material.

An intravenous formulation of the vector or composition of the invention may be in the form of a sterile injectable aqueous or non-aqueous (e.g. oleaginous) solution or suspension. The sterile injectable preparation may also be in a sterile injectable solution or suspension in a non-toxic parenterally-acceptable diluent or solvent, for example, a solution in 1,3-butanediol. Among the acceptable vehicles and solvents that may be employed are water, phosphate buffer solution, Ringer's solution and isotonic sodium chloride solution. In addition, sterile, fixed oils may be employed as a solvent or suspending medium. For this purpose, any bland fixed oil may be employed, including synthetic mono-or diglycerides. In addition, fatty acids such as oleic acid may be used in the preparation of the intravenous formulation of the invention.

The immunogenic compositions can be prepared using methodology well known in the pharmaceutical art. For example, a composition intended to be administered by injection can be prepared by combining a vector of the present invention with water so as to form a solution. A surfactant can be added to facilitate the formation of a homogeneous solution or suspension.

In an embodiment the invention relates to a host cell, comprising the vector or the immunogenic composition as described herein. The host cell may be mammalian for example human or mouse. The host cell may be transduced with the vector. The host cell may be used to produce an adenoviral stock.

In an embodiment the vector or immunogenic composition is for use in therapy. In a preferred embodiment the vector or immunogenic composition is for use in the treatment or prevention of cancer.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder: preventative treatment, that is, treatment directed to minimizing or partially or completely inhibiting the development of the associated disease, pathological condition, or disorder: and supportive treatment, that is, treatment employed to supplement another specific therapy directed toward the improvement of the associated disease, pathological condition, or disorder

The invention furthermore relates to a method of treating or preventing a cancer, comprising administering a therapeutically effective amount of the vector or composition according to the invention to a subject in need thereof.

In an embodiment the invention relates to the use of a vector or composition described herein in the manufacture of a medicament for the treatment or prevention of cancer. In an embodiment the invention relates to the use of a vector or composition described herein in the treatment or prevention of cancer.

As used herein, the term “therapeutically effective” refers to the amount of the composition used is of sufficient quantity to ameliorate one or more causes or symptoms of a disease or disorder. Such amelioration only requires a reduction or alteration, not necessarily elimination.

The present invention also provides a method of inducing an inflating memory CD8+ T cell response, comprising the step of: administering a therapeutically effective amount of the vector or composition according to the invention, to a subject in need thereof, wherein the CD8+ T cells are characterised by markers selected from the group comprising CX3CR1+, KLRG-1+, CD44+ and CD62L−.

Preferably the CD8+ T cells are characterised by the phenotype CX3CR1+, KLRG-1+, CD44+ and CD62L−. More preferably they are characterised by the phenotype CX3CR1+, KLRG-1+, CD44+, CD62L−, CD27(low), CD127(low).

The vector or immunogenic composition may be for use in the treatment or prevention of colorectal cancer, prostate cancer, oesophageal cancer, liver cancer, renal cancer, lung cancer, breast cancer, breast cancer, pancreatic cancer, brain cancer, hepatocellular cancer, lymphoma, leukaemia, gastric cancer, cervical cancer, ovarian cancer, thyroid cancer, melanoma, carcinoma, head and neck cancer, skin cancer and soft tissue sarcoma.

The vector or composition as described herein may be administered by any convenient route. The vector or composition may be administered by any convenient route, including but not limited to oral, topical, parenteral, sublingual, rectal, vaginal, ocular, intranasal, pulmonary, intradermal, intravitreal, intramuscular, intraperitoneal, intravenous, subcutaneous, intracerebral, transdermal, transmucosal, by inhalation. Parenteral administration includes, for example, intravenous, intramuscular, intraarterial, intraperitoneal, intranasal, rectal, intravesical, intradermal, topical or subcutaneous administration. In an embodiment the vector or composition is administered intravenously or intramuscularly. Compositions can take the form of one or more dosage units.

In specific embodiments, it may be desirable to administer the vector or composition of the present invention locally to the area in need of treatment such at as the site of a tumour. In another embodiment it may be desirable to administer the vector or composition by intravenous injection or infusion. The amount of the vector of the present invention that is effective/active in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances.

The compositions comprise an effective amount of the vector according to the present invention such that a suitable dosage will be obtained. The correct dosage of the compounds will vary according to the particular formulation, the mode of administration, and its particular site, host and the disease being treated. Other factors like age, body weight, sex, diet, time of administration, rate of excretion, condition of the host, drug combinations, reaction sensitivities and severity of the disease shall be taken into account. Administration can be carried out continuously or periodically.

In the therapy of cancer, the vector or immunogenic composition of the present invention, can be used in combination with existing therapies. In one embodiment, the vector or composition is used in combination with an existing therapy or therapeutic agent, for example an anti-cancer therapy. Thus, in another aspect, the invention also relates to a combination therapy comprising administration of the vector or composition of the invention and an anti-cancer therapy. The anti-cancer therapy may include a therapeutic agent or radiation therapy and includes gene therapy, viral therapy, RNA therapy bone marrow transplantation, nanotherapy, targeted anti-cancer therapies or oncolytic drugs. Examples of other therapeutic agents include checkpoint inhibitors, antineoplastic agents, immunogenic agents, attenuated cancerous cells, tumour antigens, antigen presenting cells such as dendritic cells pulsed with tumour-derived antigen or nucleic acids, immune stimulating cytokines (e.g., IL-2, IFNa2, GM-CSF), targeted small molecules and biological molecules (such as components of signal transduction pathways, e.g. modulators of tyrosine kinases and inhibitors of receptor tyrosine kinases, and agents that bind to tumour-specific antigens, including EGFR antagonists), an anti-inflammatory agent, a cytotoxic agent, a radiotoxic agent, or an immunosuppressive agent and cells transfected with a gene encoding an immune stimulating cytokine (e.g., GM-CSF), chemotherapy. In one embodiment, the vector or composition is used in combination with surgery. The vector or composition of the invention may be administered at the same time or at a different time as the other therapy, e.g., simultaneously, separately or sequentially.

In an embodiment the vector or composition is used in combination with an immunomodulatory agent. The immunomodulatory agent may be administered simultaneously, sequentially or separately with the immunomodulatory agent. In specific embodiments the immunomodulatory agent may be an immune checkpoint inhibitor, examples of immune checkpoint inhibitors include but are not limited to inhibitors of an immune checkpoint protein selected from the group consisting of CTLA-4, PD-1, PD-L1, PD-L2, TIM3, LAG-3, B7-H3, B7-H4, B7-H6, A2aR, BTLA, GAL9 and IDO.

Certain tumour types have previously been reported to be unresponsive to anti-PD-1 and anti PD-L1 monotherapies. It has surprisingly been shown herein that immunization with a minigene vector can result in enhanced tumour control when administered in combination with a checkpoint inhibitor such as an anti-PD-L1 therapy. This has been shown effective in tumour models which are known to be unresponsive to standard checkpoint inhibitor therapy. As such, in an embodiment the present vector or composition may be used in combination with a check point inhibitor for the treatment of checkpoint inhibitor unresponsive tumours.

The vector or composition of the present invention and the immunomodulatory agent may be provided as separate medicaments for administration at the same time or at different times.

In an embodiment, the vector or composition of the present invention and the immunomodulatory agent are provided as separate medicaments for administration at different times. When administered separately and at different times, either the vector or the immunomodulatory agent may be administered first. In some embodiments, both can be administered on the same day or on different days, and they can be administered using the same schedule or at different schedules during the treatment cycle.

Alternatively, the administration of the immunomodulatory agent may be performed simultaneously with the administration of the vector or immunogenic composition. Wherein simultaneous administration is used the vector or immunogenic composition and the immunomodulatory agent may be formulated as separate pharmaceutical compositions. The vector or immunogenic composition and the immunomodulatory agent may be formulated as a single pharmaceutical composition.

The vector or composition of the present invention can be administered prophylactically or therapeutically. The term “prophylactically” refers to administration intended to have a protective effect against disease. The term “therapeutically” refers to administration intended to have a curative effect.

The vector or composition of the present invention may be administered as a single dose. The dose may be provided in a prophylactic setting or a therapeutic setting. In an embodiment the single dose may be provided as a single dose unit further comprising one or more additional active ingredients, pharmaceutically acceptable carrier, diluent, excipient or adjuvant.

The vector or composition of the present invention may be administered as multiple doses. Wherein multiple doses are administered, one or more may be administered prophylactically or one or more may be administered therapeutically. Where multiple doses are administered, one or more may be administered prophylactically and one or more may be administered therapeutically. In an embodiment the vector may be administered as a “prime boost” regimen, wherein there is a first administration (a priming administration) of the adenoviral vector, followed by a second administration (a boosting administration).

Dose delays and/or dose reductions and schedule adjustments are performed as needed depending on individual patient tolerance to treatments.

Wherein the immunogenic composition comprises at least two vectors and wherein the vectors encode different epitopes as described above, there may be synergy between the vectors. As such, each of the vectors may be administered at a sub-optimal dose. The term “sub-optimal” dose refers to a dose level that it is not intended to fully remove or eradicate the tumour, but nevertheless results in some tumour cells or tissue becoming necrotic. The skilled person will be able to determine an appropriate dose required in order to achieve this, depending on factors such as; age of the patient, status of the disease and size and location of tumour or metastases

In an embodiment there is provided a method of producing the vector are described above, comprising the steps of:
i) synthesising the nucleic acid sequence encoding the epitope, as a sense and antisense primer,
ii) cloning the nucleic acid sequence encoding epitope sequence into a first plasmid,
iii) cloning the sequence comprising the nucleic acid sequence encoding epitope into a second plasmid comprising the adenoviral DNA.

Suitable cloning methods are known within the art, examples of cloning methods include but are not limited to, restriction ligations methods, Gateway cloning, Gibson assembly, ligation independent cloning. The person skilled in the art will be able to determine a suitable method to clone the sequence into the plasmid. The cloning method to introduce the nucleic acid sequence encoding epitope sequence into the first plasmid may be the same or different from the cloning method used to. In an embodiment the cloning method to introduce the nucleic acid sequence encoding epitope sequence into the first plasmid is selected from restriction ligations methods, Gateway cloning, Gibson assembly, ligation independent cloning. In an embodiment the cloning method to introduce the nucleic acid sequence encoding epitope into the second plasmid comprising the adenoviral DNA is selected from restriction ligations methods, Gateway cloning, Gibson assembly, ligation independent cloning.

In an embodiment, step iii) comprises cloning the sequence comprising the nucleic acid sequence encoding the epitope into a second plasmid comprising the adenoviral DNA, wherein the sequence comprising the nucleic acid sequence encoding the epitope also comprises additional features selected from the group comprising a translation initiation sequence, a promotor, a termination sequence, a polyadenylation sequence.

In an embodiment the method of producing the vector comprises the steps of:
i) synthesising the nucleic acid encoding the epitope, as a sense and antisense primer,
ii) allowing the sense and antisense primers to anneal,
iii) digesting the annealed primers with appropriate restriction enzymes to allow insertion into a donor plasmid, and
iv) transferring the donor plasmid into a second plasmid comprising the adenoviral DNA.

Appropriate restriction enzymes and sites will be known to the skilled person. It is within the capability of the skilled person to design appropriate restriction sites within the sense and anti-sense primers to allow insertion into the donor plasmid.

The epitope that is encoded is a cancer specific CD8+ and/or CD4+ T cell epitopes. Multiple cancer specific epitopes have been determined and are known in the art. It would be possible for the skilled person to select an appropriate epitope to be encoded within the vector. Further methods for identifying cancer specific epitopes are known in the art include bioinformatics approaches, transcriptome analysis and in silico methods.

The second plasmid which encodes the adenoviral vector may comprise any of the following features. The adenoviral vector may comprise enhancer and promoter regions for example a strong promoter such as a CMV promoter, an RSV promoter, an EF1α promoter. In a preferred embodiment the vector comprises a CMV promoter. The vector may comprise a TATA box. In an embodiment the vector comprises a translation initiation sequence, for example a Kozak sequence. A Kozak sequence has the consensus sequence (gcc) gccRccAUGG. In an embodiment the vector comprises a termination sequence and/or a polyadenylation sequence. The adenoviral vector may be a Species C serotype such as Ad1, 2, 5 and 6 serotypes. In a preferred embodiment the adenoviral vector is a human serotype 5 (AdHu5). It may be preferable for the adenoviral vector to be modified for example to reduce the immunogenicity and improve biosafety of the vector. As such, the adenoviral vector may be replication-incompetent. The adenoviral vector may lack the E1 and E3 proteins.

Transferring the donor plasmid into the second plasmid may be performed by any method, for example ligation methods.

In an embodiment of the present invention there is provided a kit comprising the vector or immunogenic composition as described herein, one or more additional active ingredients, pharmaceutically acceptable carrier, diluent, excipient or adjuvant, and optionally instructions for use.

The additional active agent may include checkpoint inhibitors, antineoplastic agents, immunogenic agents, attenuated cancerous cells, tumour antigens, antigen presenting cells such as dendritic cells pulsed with tumour-derived antigen or nucleic acids, immune stimulating cytokines (e.g., IL-2, IFNa2, GM-CSF), targeted small molecules and biological molecules (such as components of signal transduction pathways, e.g. modulators of tyrosine kinases and inhibitors of receptor tyrosine kinases, and agents that bind to tumour-specific antigens, including EGFR antagonists), an anti-inflammatory agent, a cytotoxic agent, a radiotoxic agent, or an immunosuppressive agent and cells transfected with a gene encoding an immune stimulating cytokine (e.g., GM-CSF).

The pharmaceutical acceptable carrier, diluent, excipient or adjuvant may include: sterile diluents such as water, saline solution, preferably physiological saline, Ringer's solution, isotonic sodium chloride, fixed oils such as synthetic mono or digylcerides, polyethylene glycols, glycerin, or other solvents: antibacterial agents such as benzyl alcohol or methyl paraben: and agents for the adjustment of tonicity such as sodium chloride or dextrose.

In an embodiment the present invention relates to a method for inducing a T cell immune response in an animal against a cancer specific CD8+ and/or CD4+ T cell epitope, comprising contacting a cell with the vector or immunogenic composition as described herein.

The cell may be contacted with the vector or composition in an in vitro manner, in an ex vivo manner, or in an in vivo manner. Wherein the cells are contacted with the vector or composition either in vitro or ex vivo, the cells may then be administered to a subject.

The T cell immune response may comprise an inflating memory CD8+ T cell response.

In another aspect, the invention provides a vector as set out in the examples and/or accompanying figures.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification are incorporated herein by reference in their entirety.

The invention is further described in the non-limiting examples.

### EXAMPLES

### Example 1: Single AdHu5 Construct Encoding the Dominant AH1 Epitope, a CD8 Epitope Identified in CT26 Colorectal Carcinoma is Immunogenic

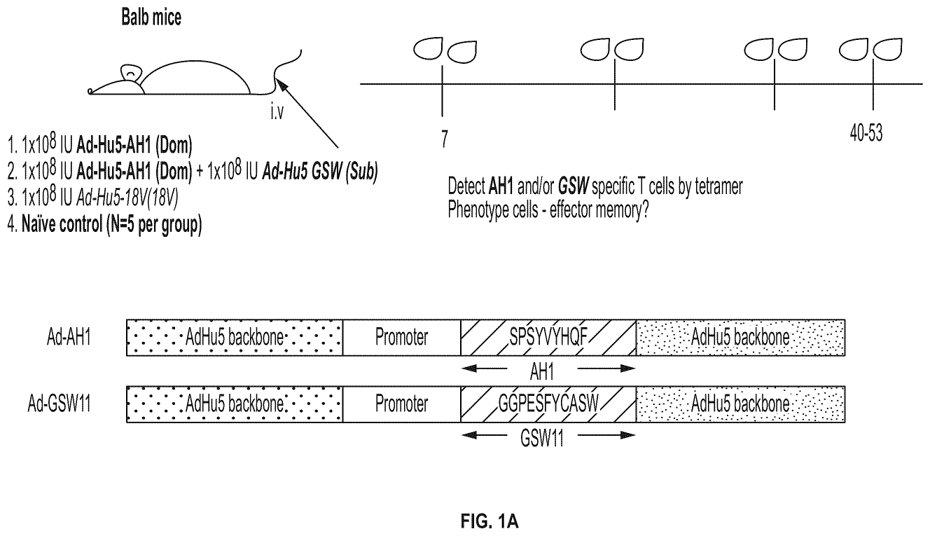

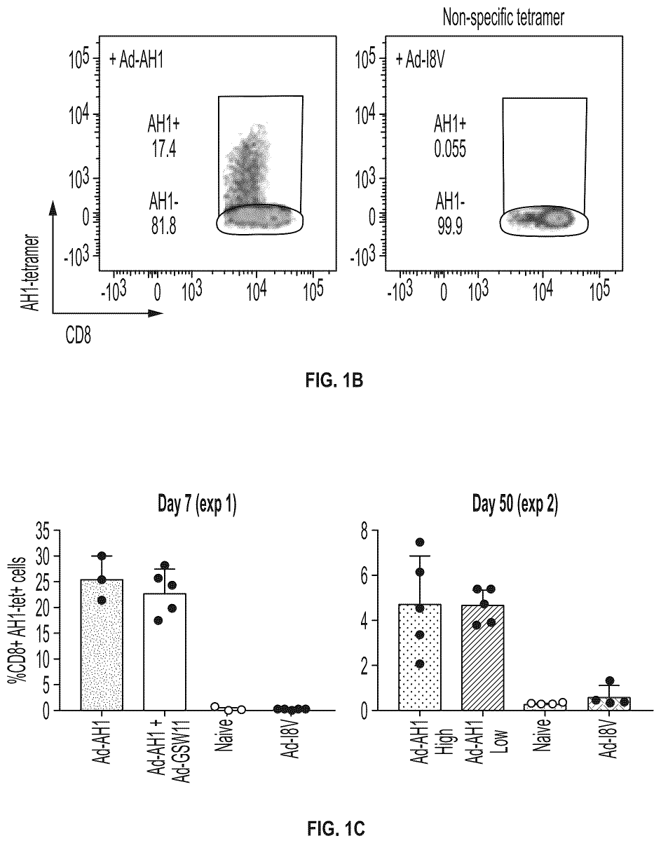

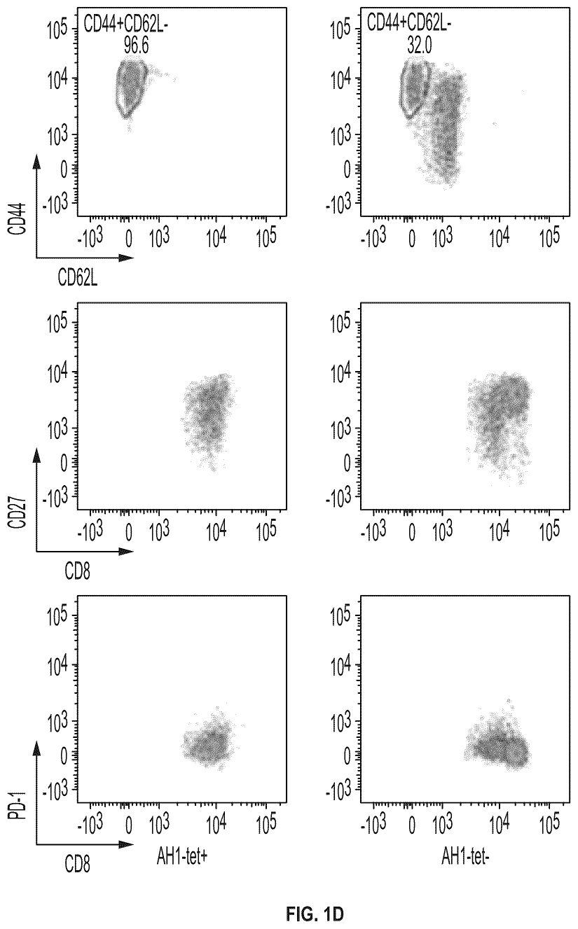

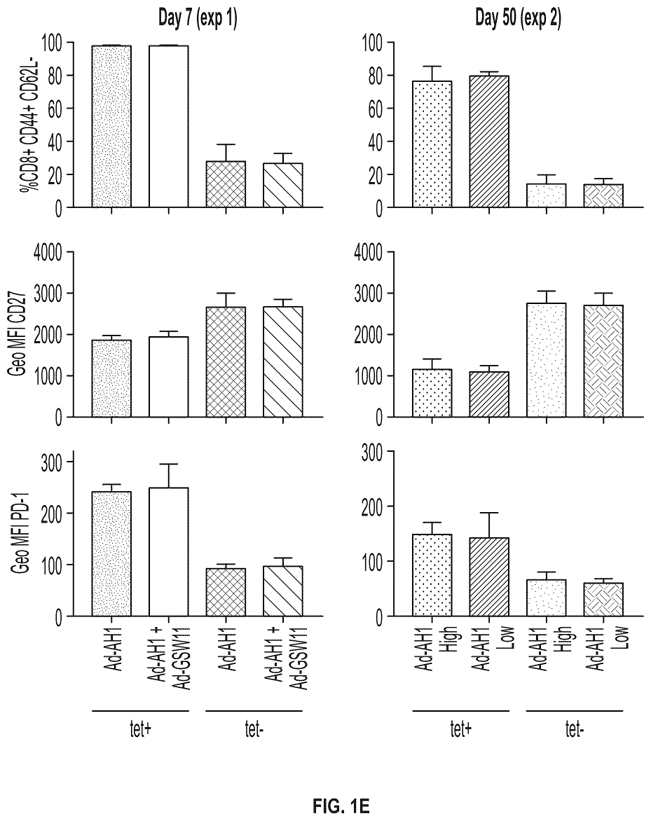

A series of experiments in a mouse cancer model with an endogenous neoantigen was performed to investigate whether minigene vaccination is able to raise a T cell response against an endogenous T cell epitope. We utilized the CT26 murine colorectal carcinoma model, where the peptide sequence SPSYVYHQF (termed AH-1 SEQ ID NO:1) was derived from the protein of the murine leukaemia virus (MuLV env gp70₄₂₃₋₄₃₂), an endogenous retrovirus which is recognized by Balbc mice in a H2-DL-restricted manner. A minigene encoding a “cryptic” CD8 T cell epitope, GGPESFYCASW (from MuLV env gp90₁₄₇₋₁₅₈, termed GSW11 SEQ ID NO:3) was also tested. This H-2D^{d}-restricted epitope does not induce a CD8 T cell response in healthy immunocompetent BALB/c mice—although it is also derived from MuLV it is encoded in a different open reading frame to AH-1. Furthermore, it does not stably bind the D^{d} MHC molecule as it does not conform to the canonical peptide motif and as such has a very rapid half-life of stabilization of 20 mins before it is lost from the cell surface. By contrast AH-1 has a half-life of 60 mins: consequently, CD8 T cell-specific responses to GSW11 only develop when regulatory CD4 T (Treg) cells are systemically depleted (James et al., 2010 J. Immunol. 185:5048-5055) leading to a very high level of activation of antigen presenting cells (Shevach., 2009 Immunity 30(5):636-45). This was included to examine whether responses against such unstable “cryptic” epitopes could be raised by minigene immunization in immunocompetent animals without requiring systemic Treg depletion. The two epitopes were constructed as separate minigenes on the AdHu5 backbone as previously described (FIG. 1A).

Injection of BALB/c mice with minigene vector AdHu5−AH1−MG, at a dose of 1×10⁸ or 1×10⁷ Infectious Units (IU) for Ad-AH1 Low, induced AH1-specific CD8+ T cells in the blood as detected by AH1-tetramer staining (FIG. 1B) at approximately 25% of total CD8+ T cells at day 7 post-vaccination (FIG. 1C, left). This level gradually decreased over time to ˜5% at day 50) (FIG. 1C, right) and ˜2.5% at day 80 post-vaccination (data not shown). Similar percentages are observed following Ad-minigene vaccination in C57BL/6 mice, albeit plateau at higher levels. No AH1-specific responses could be detected in naïve mice nor mice vaccinated with an AdHu5 minigene encoding an irrelevant epitope (I8V, an epitope derived from β-galactosidase—a bacterial enzyme) (FIG. 1C left and right).

GSW11-specific responses could not be detected by GSW11-tetramer staining or GSW11-peptide stimulation indicating that such “cryptic”, unstable epitopes are not able to generate a CD8 T cell response in immunocompetent animals. Nevertheless, it is interesting to note that there was no difference in the magnitude of the AH-1+ tetramer response in groups immunized with only AdHu5-AH-1 versus animals immunized with both minigenes, AdHu5-AH-1+AdHu5-GSW11, (FIG. 1C, left), indicating that co-delivery of an AdHu5 minigene construct encoding a non-immunogenic epitope did not interfere with the induction of the AH-1 specific response.

While the magnitude of AH1-specific CD8+ T cells decreased over time, their phenotype remained stable-similar to that observed in C57BL/6 mice15, have an effector memory phenotype (CD44+ CD62L−, FIG. 1D and 1E top row), express lower levels of CD27 (FIG. 1D and 1E, middle row) and higher levels of PD-1 (FIGS. 1D and 1E, bottom row) compared to the tetramer negative (tet−) population in the blood from the same mice (FIGS. 1D and 1E). In addition, high levels of CX3CR1 and low levels of CD127 were also detected within this population. By day 50, a slight loss of the effector memory phenotype was observed, together with some upregulation of the activation marker CD27 and PD-1 (FIG. 1E, left panels, top, middle and bottom respectively).

### Example 2: AdHu-5 Minigene Immunization Delays CT26 Tumour Growth in Prophylactic and Therapeutic Immunization Models

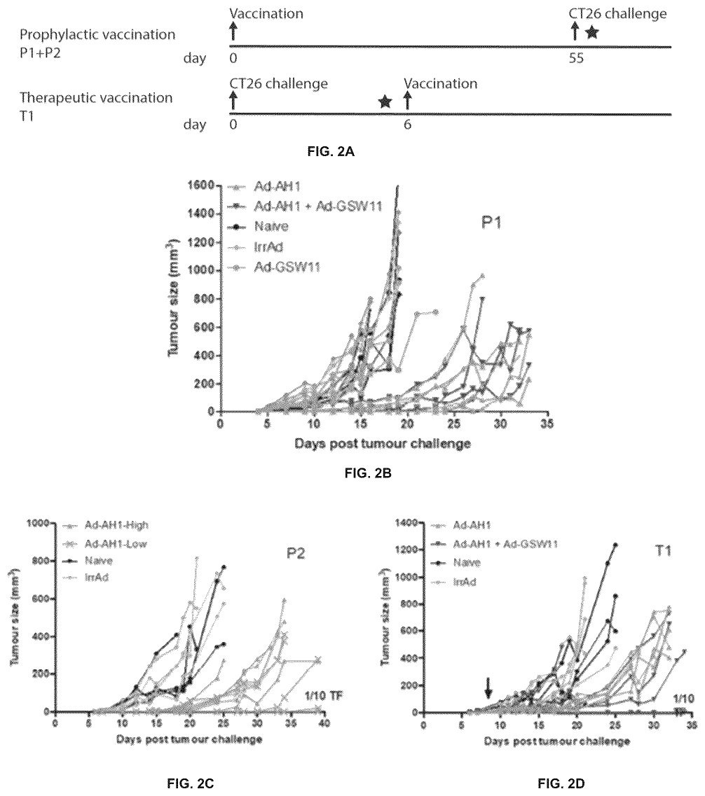

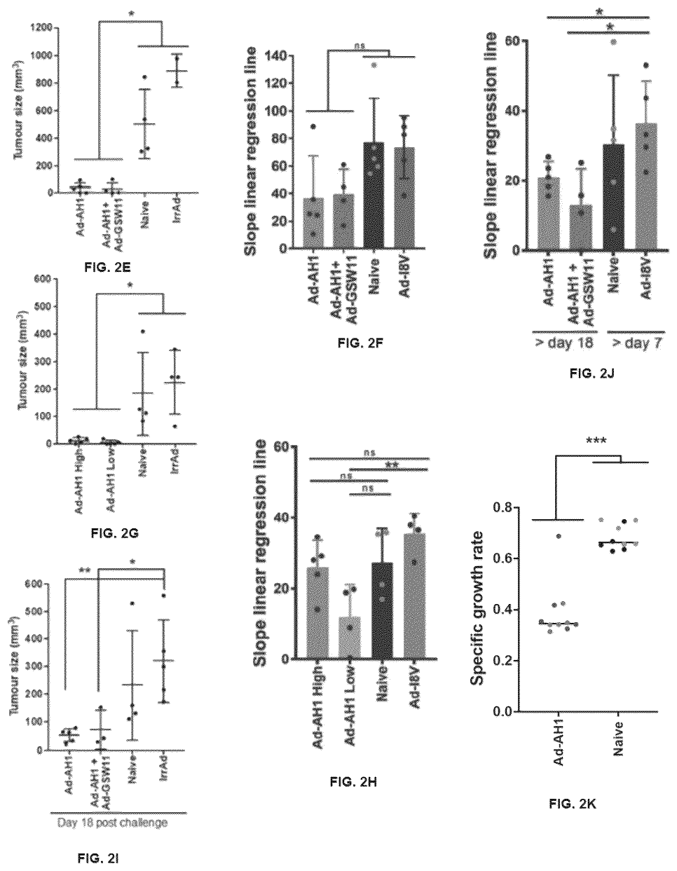

To measure the protective efficacy of this prophylactic immunization regimen. the immunized animals were injected subcutaneously (s.c) with CT26 tumour cells 5 days post-Ad-AH1-vaccination (FIG. 2A, B-C). All animals immunized with Ad-AH1 significantly suppressed tumour growth, with complete remission in one of the animals challenged (1/15) (FIGS. 2B and C). As expected from the immunogenicity data, no protection was seen in groups immunized only with Ad-GSW11 (FIG. 2B-P1), irrelevant minigene or non-immunized (FIG. 2B-P1). Interestingly, marginally better control was seen in the group vaccinated with a low dose of Ad-AH1 (1×10⁷ IU)¹⁷ (FIG. 2C-P2) (although not statistically significant). Significant difference in tumour sizes was observed at day 18 post-tumour challenge, between Ad-AH1 vaccinated and control mice (FIGS. 2E and 2G). In addition, the rate at which tumours grow (determined by the slope of the linear regression line fitting the curve) is lower in the Ad-AH1 vaccinated mice compared to control mice from the day tumours start to grow (FIGS. 2F, 2H and 2K). In conclusion, Ad-AH1 vaccination pre-and post-CT26 tumour challenge delays tumour growth.

We next tested the minigene constructs in a therapeutic challenge model (FIG. 2D). Groups of mice were injected s.c with tumour cells, and then 6 day later injected i.v. with Ad-AH1 (FIG. 2A and FIG. 2D). As before, immunization with Ad-AH-1 delayed tumour growth-Ad-AH1-immunized mice had significantly smaller tumours compared to naïve and irrelevant AdHu5-immunized animals at Day 18 post-immunization (FIGS. 2I and 2J) with an animal remaining tumour-free (1/10).

### Example 3: AdHu5-AH1 Minigene Immunization Alters the Phenotype of Specific CD8+ T Cells

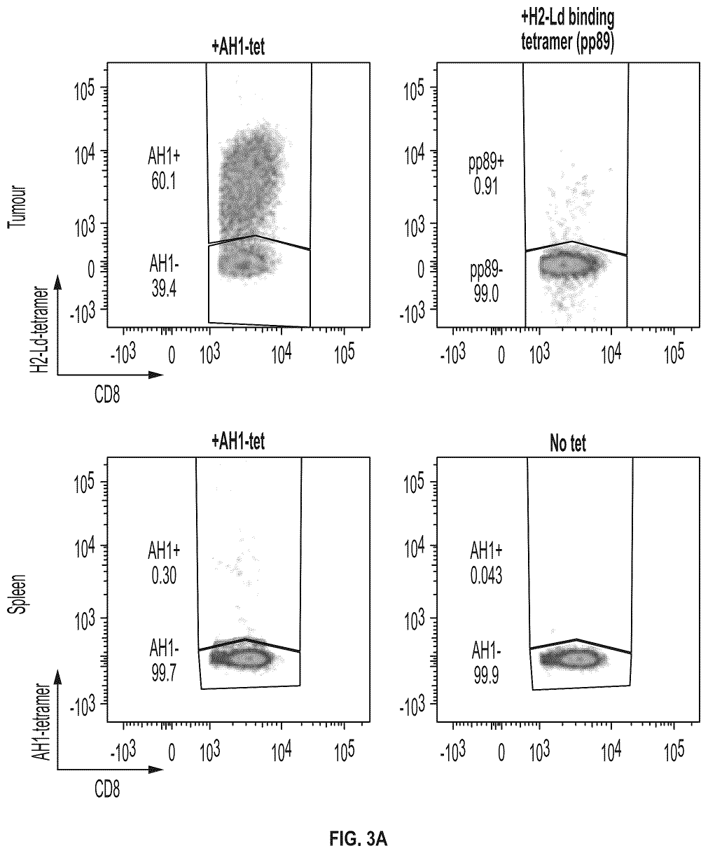

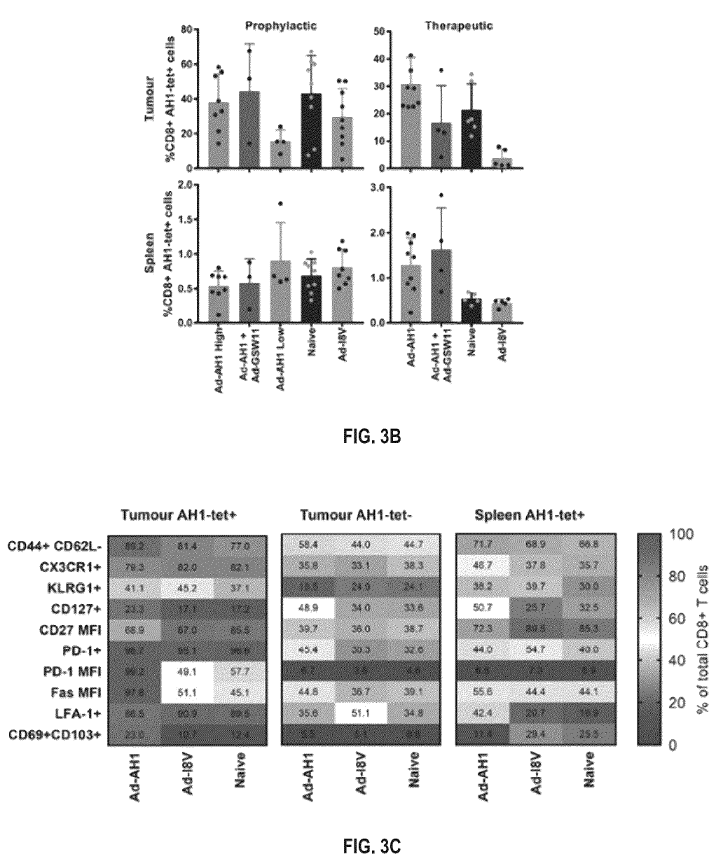

The tumour-bearing mice were culled when the humane endpoint was reached. At that point, the magnitude of AH-1-specific CD8 T cells in tumours (TIL) and spleen was determined. AH1-tetramer staining (FIG. 3A, upper panel), showed high levels of AH1-specific CD8+ T cells in tumours from vaccinated mice as well as in control mice (FIG. 3B, upper row, left and right). Staining with the complete panel of fluorochrome-conjugated antibodies together with a different/irrelevant H2-Ld-binding tetramer (recognizing the MCMV pp89 epitope), did not show any positive cells (FIG. 3A), confirming that the high levels of AH-1 tetramer cells are not a consequence of autofluorescence or non-specific binding of the H2-Ld tetramer. It is worth noting however that at this stage, loss of tumour control had occurred.

At the late stage of prophylactic immunization, a population of AH-1 specific CD8 T cells were detected in the spleen (FIG. 3B, bottom row, left) in all groups, with little difference between the groups. By contrast only animals immunized with Ad-Hu5 AH-1 after tumour challenge developed elevated levels of AH-1 tetramer positive cells in the spleen (FIG. 3B, bottom row, right), suggesting that immunization may boost the levels of tetramer positive cells in other compartments.

Phenotypic analysis showed that in the spleen, these AH1-specific CD8+ T cells from Ad-AH1 vaccinated mice are mostly effector memory cells (CD44+ CD62L−) which upregulate CX3CR1, CD127, Fas, and LFA-1, and downregulate CD27 and Trm cell markers (CD69+CD103+) (FIG. 3C, right panel). By contrast, AH1-specific CD8+ T cells in the tumours expressed a different phenotype, becoming highly upregulated but exhausted, as evidenced by high levels of PD-1 (FIG. 3C, left panel). PD-1 upregulation is likely due to extensive TCR stimulation, as the levels of PD-1 on the other CD8 T cells in the TIL was not as high (FIG. 3C, middle panel). All indicated markers are elevated except for CD127 which is downregulated, and CD27 and CD69+CD103+ which remain the same in both tetramer positive and non-tetramer positive TIL (FIG. 3C, left and middle panels). Therefore, immunization appears to alter the level of tetramer positive cells in lymphoid compartment and skew the phenotype towards one of effector memory in the TIL.

### Example 4: The Percentage of Regulatory T Cells Appears to be Lower in Tumours from Ad-AH1 Vaccinated Mice Compared to Control Mice While Trms are Increased in TILs After Ad-AH1 Immunization

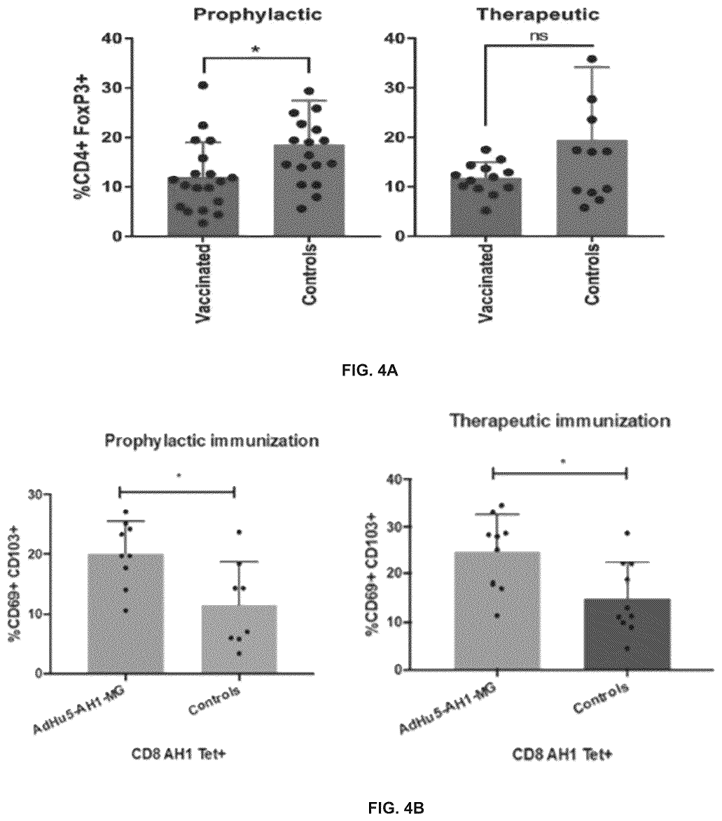

To determine whether immunization results in other alterations in the tumour microenvironment, the levels of Treg and AH-1-specific resident memory T cells (Trm) were measured. We found that within the CD4 T cell compartment, the proportion of Tregs (CD4+FoxP3+) cells were lower in tumours from Ad-AH1 vaccinated compared to control groups (naïve and irrelevant-Ad-immunized) (FIG. 4A).

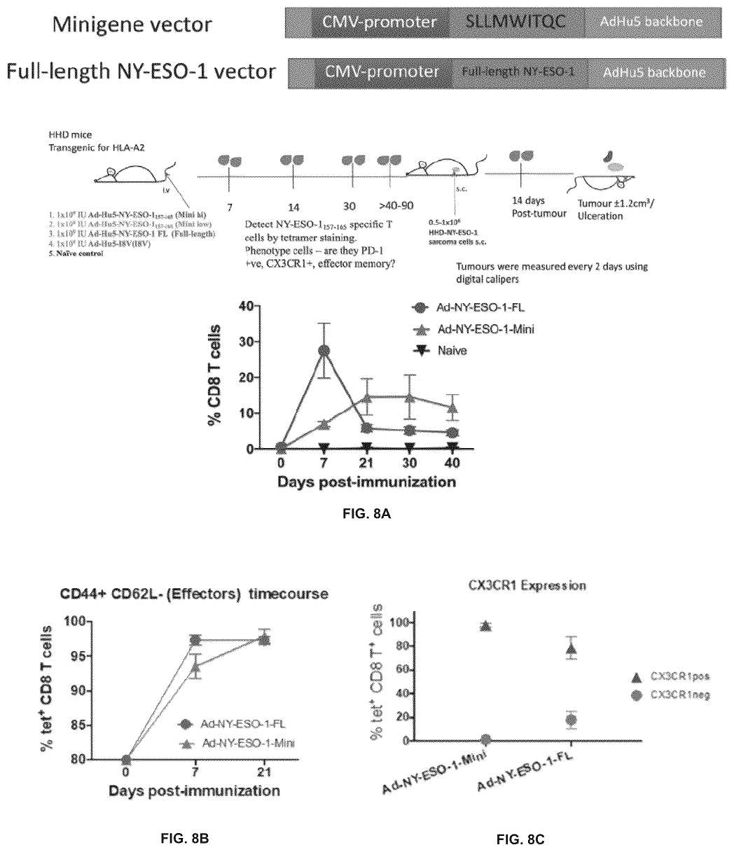

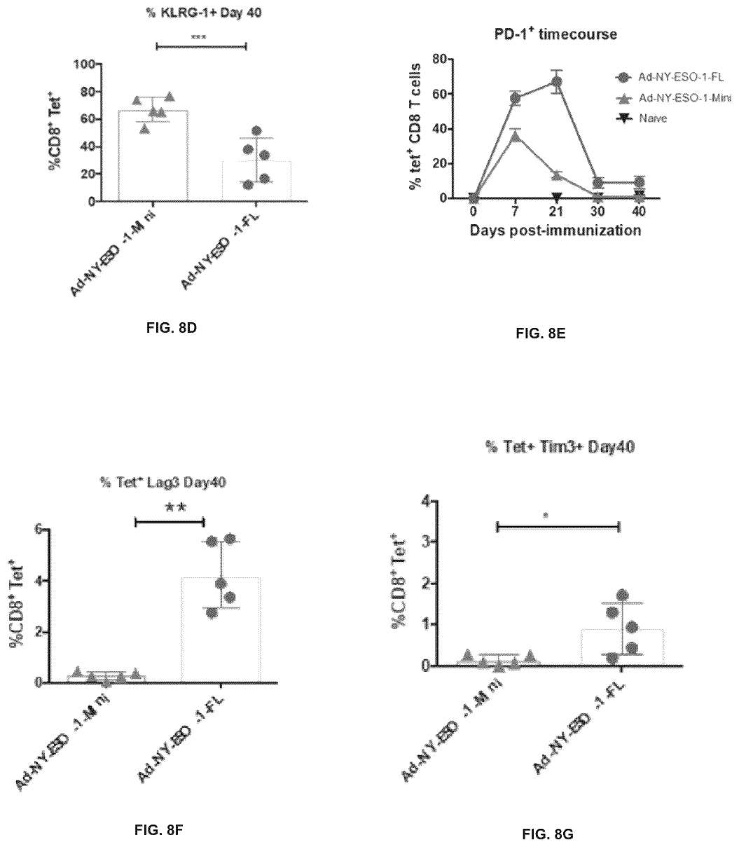

Recently it has been reported that antigen-specific CD8 T cells expressing Trm phenotype exerted superior tumour control. In line with this, we found that adenoviral vector minigene immunization increases the percentage of AH1+CD103+CD69+ Trm in the TIL. This is statistically significant when immunized groups are combined and compared against negative control (IrrAd and Naïve) groups. In the naïve group, despite the presence of large populations of AH1+ tetramers (FIG. 8A), few displayed the Trm phenotype of CD103+CD69+CD62L low, CD44hi (FIG. 4B). This increase was evident in both prophylactic and therapeutic immunization settings (FIG. 4B). Taken together, in this cancer model, minigene immunizations appears to alter the tumour microenvironment towards favouring recognition and killing of tumour cells.

### Example 5: While Antigen-Specific CD8 T Cells in the TIL are not Responsive to Cognate Peptide, Splenocytes from the Cognate Animals Retain their Functionality

The high percentage of AH1-specific CD8+ T cells detected in the tumour by AH1-tetramer staining (FIG. 3A) indicates the number of cells that express the AH1-specific TCR. However, this does not prove whether TCR signalling and T cell activation occurs upon interaction with AH1-peptide. Therefore, we stimulated single cells from spleen and tumour with (1) AH1-peptide to measure effect of TCR signalling (2) PMA/IO to measure non-specific activation. The production of the pro-inflammatory cytokine interferon gamma (IFNγ) was used as a read-out. Splenocytes from the corresponding mice were also stimulated with cognate peptide and PMA/IO.

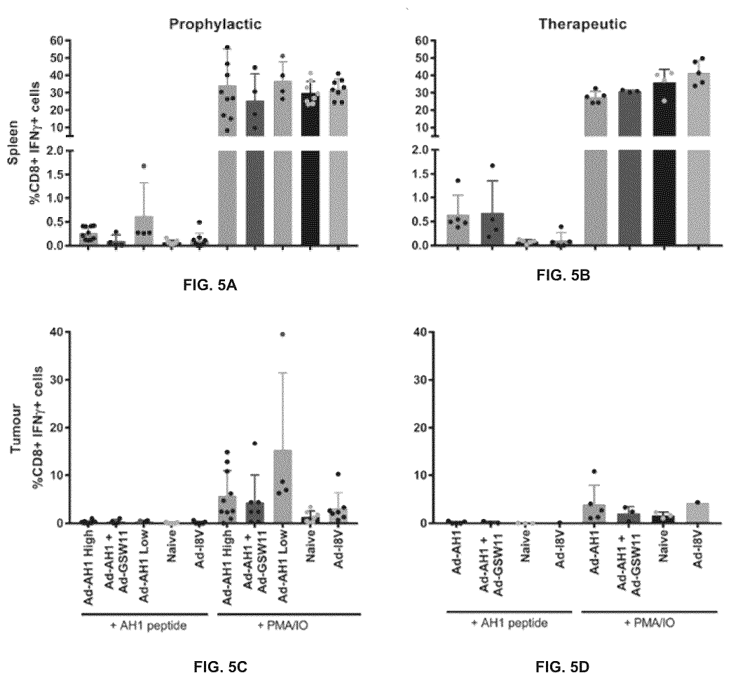

As shown in FIGS. 5A and 5B, CD8+ splenocytes from minigene-immunized groups are able to respond (i.e. produce IFNγ) when stimulated with AH1-peptide ex vivo from both prophylactic and therapeutic vaccinated mice and very little responses were recorded from the non-AH1-immunized splenocytes. By contrast, there was very low/no cytokine production observed in the TILs of the corresponding immunized animals. PMA/IO stimulation also induced very little IFN-gamma production from the immunized TILs, with even lower levels observed in the non-AH-1 immunized animals. Taken together, the results suggest that at late timepoints, antigen-specific CD8 T cells in the tumour are dysfunctional although the similar antigen-specific CD8 T cells in other compartments maintain their functionality. Furthermore, the dysfunction in the TILs is likely intrinsic as the cells were also not able to respond to PMA/IO stimulation, which does not require intact antigen presenting cells. Finally, these results also show that while minigene immunizations raises a population of IFN-gamma producing antigen-specific cells in other compartments, e.g. the spleen, this does not occur when antigen-specific cells are raised in response to the tumour cells.

### Example 6: Minigene Immunization Induces a Population of AH1-Specific CD8 T Cells in the Periphery that Slows Down Tumour Growth

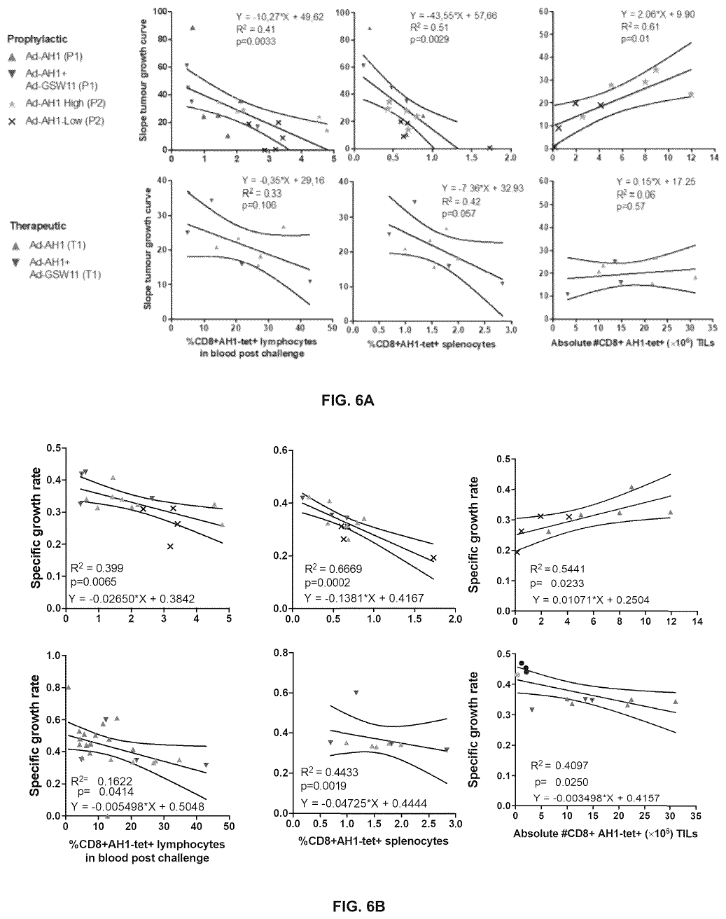

An additional effect of immunization was uncovered when the growth rates of the tumours were calculated. Growth rates were determined by calculating the slope of the linear regression line fitting the curve taken from the day the tumours show clear tumour growth (day 7 post-implantation for negative controls and day 18 post-implantation for AH1-immunized animals) (FIG. 6A). Alternatively, specific growth rates were calculated using the same raw data (FIG. 6B). We found that even when tumour escape occurred, the tumours grew at a slower rate compared to controls, suggesting that the selection pressure exerted by the inflating cells may have resulted in outgrowth of less fit tumour population. Alternatively, the tumours are actually dividing at the same rate as controls but a proportion of them are always cleared by the inflating anti-AH1 CD8 T cells. When the growth rates of the tumours (from FIG. 2D) were plotted against the percentage of tetramer positive cells in TIL or spleen after tumour challenge, a strong inverse correlation was observed between the tumour growth rate and the percentage of AH1 tetramer+ splenocytes (FIG. 3B) from both prophylactic and therapeutic challenge studies, indicating that higher levels of these cells result in better control of tumour growth

### Example 7: Immunization with AdHu5-AH1-Minigene Construct May Confer Better Tumour Control Compared to Immunization with AdHu5-gp90FL

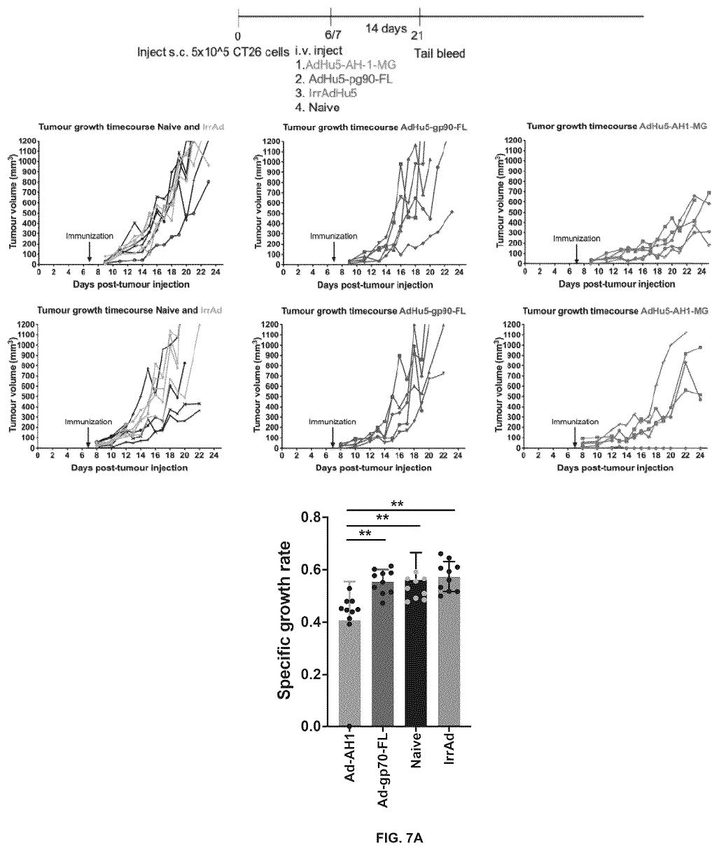

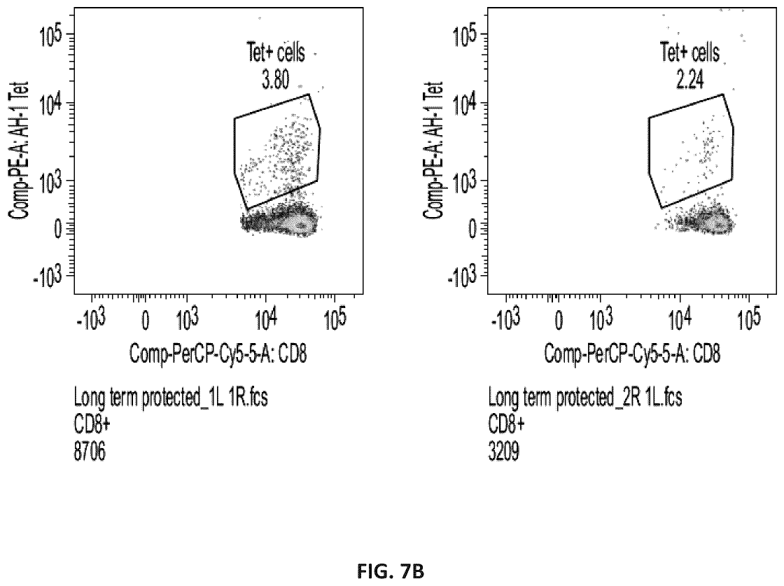

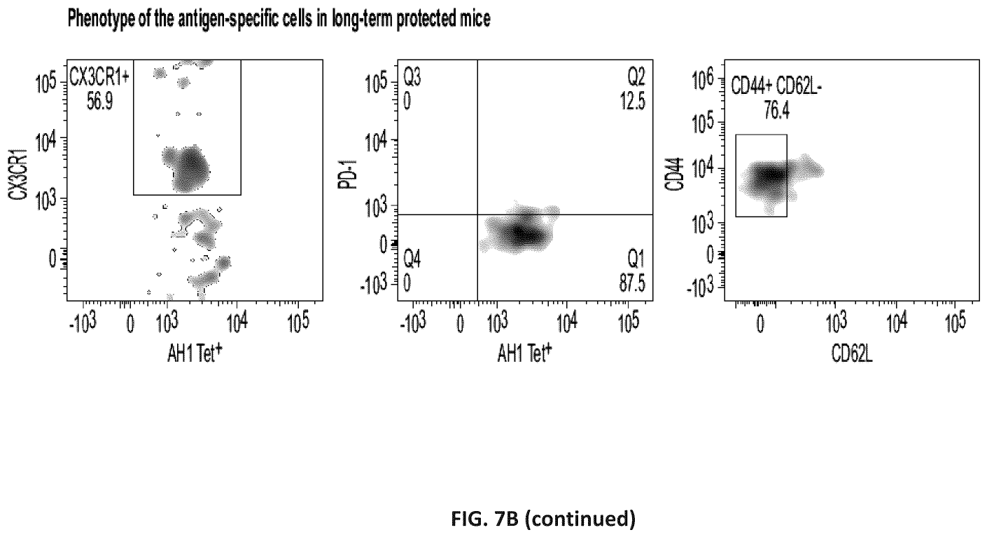

The protection afforded by adenoviral constructs encoding the dominant CD8 T cell epitope and a similar construct encoding the full-length protein gp90), from which the epitope is derived from, was compared in the therapeutic immunization experiment. Here, minigene constructs were found to exert better control compared the AdHu5-gp90)-FL as evidenced by statistically significant lower growth rates of the tumours (FIG. 7A). The blood from mice which cleared the tumours (from FIG. 2B) were sampled approx. 6 months post challenge and a population of AH1 Tet+ cells continued to be detected in circulation, indicating a functional CD8 T cell response is present long-term (FIG. 7B).

### Example 8: Immunization with an AdHu5-NY-ESO-1 (157-165) (SEQ ID NO:2), a HLA: A2-Restricted CD8 T Cell Epitope Leads to Development of HLA:A2-Restricted Inflating Memory Response

A minigene construct expressing the dominant HLA-A2 restricted epitope from the cancer testis antigen NY-ESO-1 was generated (FIG. 8A) by inserting the epitope under the control of the CMV promoter on a replication-deficient AduHu5 backbone with the E1 and E3 genes deleted. A control adenovector containing the full-length NY-ESO-1 was also constructed. These were injected i.v. into transgenic HHD mice expressing the HLA-A2 antigen, on a C57BL/6 background. In HHD mice, there is also a knock-out of H-2 Db and the mouse beta-2-microglobulin (b2m) (as well as the HLA-A2 HHDb2m hybrid molecule). This results in only HLA-A2 as the MHC class 1. Their CD8 T cell responses to the epitope NY-ESO-1 was followed in the blood by tetramer staining. As shown in FIG. 8A, mice immunized responded to either constructs with a population of tetramer specific cells which were measurable at Day 7, In the majority of mice immunized with full-length NY-ESO-1 protein, the responses diminished by day 21 and remained at low but detectable levels (approx. 2-5%) for the duration of the experiment, although there were some mice which displayed high levels (up to 20%) of this response even at the late timepoints. By contrast, the majority of mice immunized with the minigene construct displayed consistently elevated levels of the tetramer positive CD8 T cells at subsequent timepoints. This was consistent with previously reported kinetics after immunization with minigene vectors.

The tetramer positive cells were phenotyped, and were found to display inflating cell phenotypes, being predominantly effector memory (CD44+ CD62L−, FIG. 8C), terminally differentiated, expressing KLRG1-hi (FIG. 8D), and CX3CR1+ (FIG. 8E). These cells were also PD-1 low in the later stages and interestingly appeared to express lower levels of PD-1 compared to tetramer positive cells which were generated by immunization with the full-length construct (FIG. 8F). Levels of other exhaustion markers such as Tim-3 and Lag-3 were also lower in minigene-induced Tetramer+ CD8 T cells compared to their full-length induced counterparts (FIGS. 8G and H). Taken together the data show that CD8 T cell peptide epitopes on minigene constructs are able to be processed and loaded onto human HLA-A2 antigens, which are then able to prime and generate an inflating CD8 T cell response. Furthermore. these responses are large and durable with very low/no expression of checkpoint inhibitors even at late timepoints post immunization.

### Example 9: Immunization with AdHu5-NY-ESO-1 (157-165) Controls Tumour Challenge

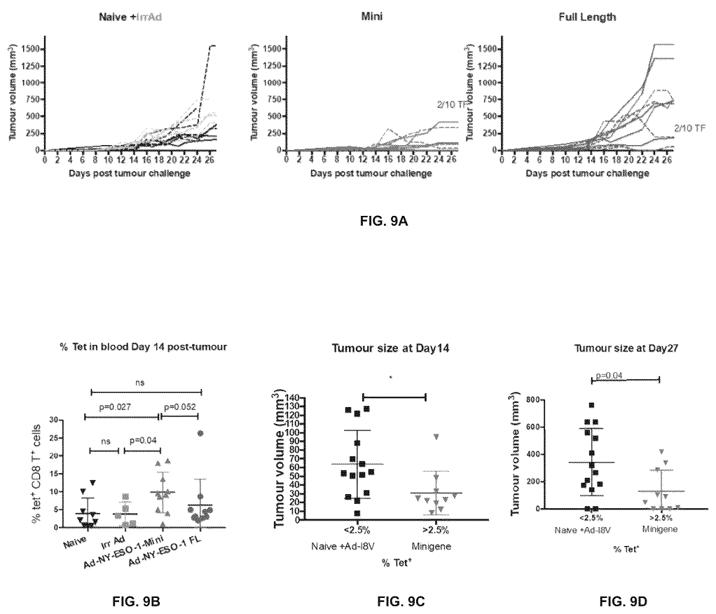

To determine whether these responses are able to control tumours, the mice were subcutaneously injected (s.c.) with a high number of sarcoma cells (0.5-1×10⁶ cells) derived from HHD mice which were stably transfected with NY-ESO-1 protein. The tumour growth was tracked. The results indicate the mice immunized with the AdHu5-NY-ESO-1 minigene was able to delay tumour growth at early and late timepoints (FIG. 9A) with 2/10 animals showing complete clearance of the tumour. Additionally, this control was observed in both high (solid lines) and lower (dashed lines) dose challenge. By contrast, mice immunized with FL vector were able to control tumour growth at the lower challenge dose but failed to do so at the higher cell concentration (solid lines). It is worth noting that as the mice are only transgenic for HLA-A2, NY-ESO-1 being a human protein would likely be immunogenic and recognized by naïve mouse CD4 and CD8 T cells upon tumour challenge. Blood taken two weeks after tumour challenge was analysed for the presence of Tet+ cells. All groups developed a detectable circulating tet+ response 14 days after tumour challenge, with the MG-immunized group displaying the largest magnitude (FIG. 9B). Animals that had more than 2.5% of tet+ cells in circulation prior to tumour challenge exerted better control of tumour growth at the early and late timepoints (FIGS. 9C and D). This correlation was not observed in animals immunized with FL vectors. The data from this part of the experiment indicates that a single priming immunization with minigene vectors is able to confer long-lived protection against tumour challenge.

### Example 10: Immunization with AdHu5-NY-ESO-1 (157-165) Leads to a Population of Antigen-Specific T Cells in the Spleen

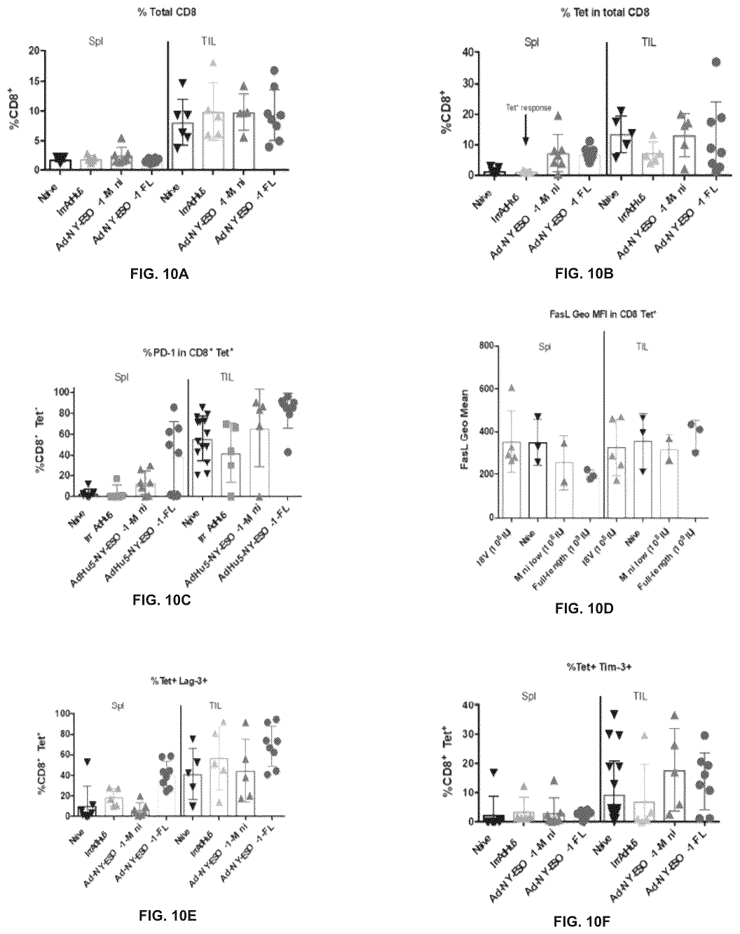

Animals were culled when they reached their endpoint which was either when the tumour size was approaching 1300 mm³ or when ulcers developed which did not improve after 48 hours. Tumours were removed between day 17-29 for naïve groups, days 28-29 for MG, days 26-29 for FL and days 22-29 for irrelevant Ad groups. At that point, the lymphocytes were isolated from the tumours and spleens to investigate whether immunization altered the composition of the tumour immune microenvironment and functionality of the tumour-specific cells. Splenocytes and tumour-infiltrating lymphocytes (TILs) were isolated and TILS from all groups were found to contain similar levels of CD8 T cells (FIG. 10A). The levels of splenic CD8 T cells were slightly elevated in the MG-immunized group, but this did not reach statistical significance. NY-ESO-1 tet+ cells were detected in the TILS of all groups with no statistical difference in the percentage of Tet+ TILs between immunized and non-immunized groups (FIG. 10B). A difference in the percentage of Tet+ splenocytes was observed however, with higher levels in MG and FL-immunized animals compared to unimmunized animals. The tumours were removed because they had reached their endpoint and at this timepoint, expression of the checkpoint inhibitor PD-1 was found to be elevated in tet+ TILs in all groups (FIG. 10C). Fas was upregulated in splenocytes and also TILs of all the groups (FIG. 10D) indicating activation of the Tet+ cells. Interestingly, CD8 Tet+ splenocytes from MG and FL-immunized animals expressed higher levels of PD-1. with FL-immunized animals showing the highest expression of PD-1 on splenocytes, FL-immunization also resulted in higher levels of Lag-3 on the splenocytes and TILs. Lag-3 and Tim-3 was not upregulated on the Tet+ splenocytes of the other conditions: but was detected in Tet+ TILs at similar levels in all groups (FIGS. 10E and F).

### Example 11: CX3CR1 is Upregulated in the Antigen-Specific Splenocytes after Immunization with AdHu5-NY-ESO-1 (−)

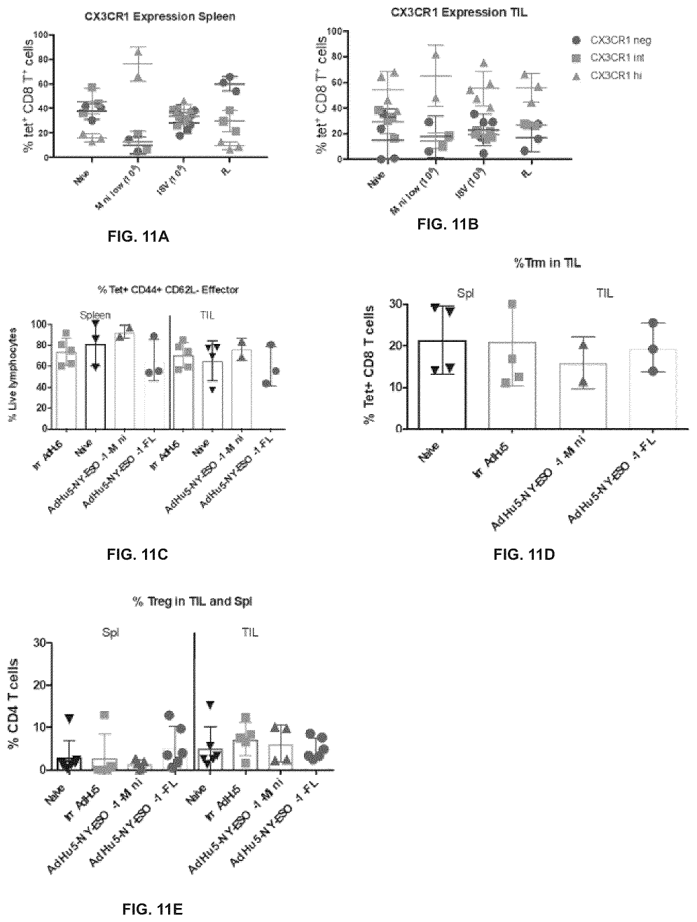

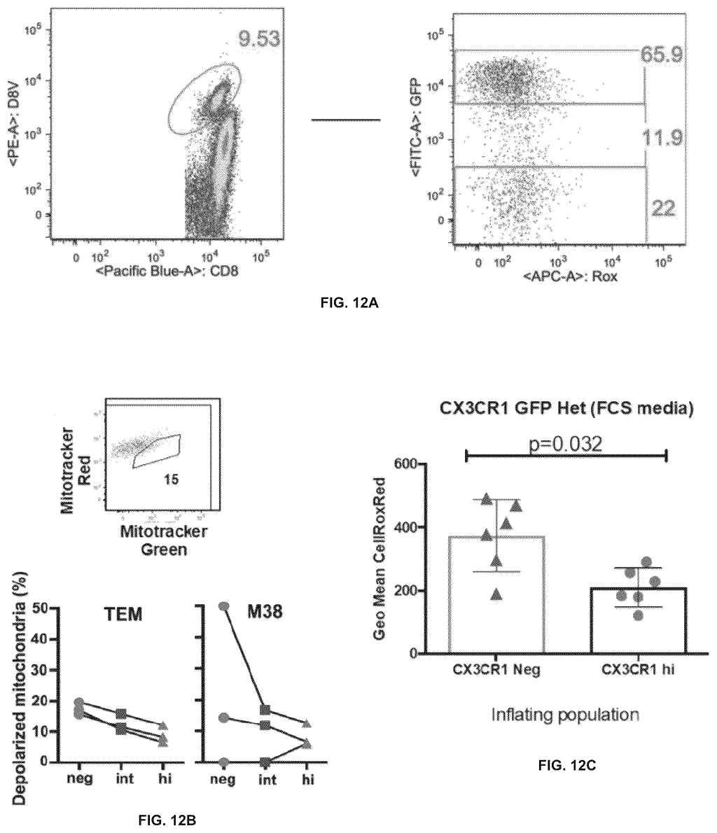

TILs and splenocytes were further characterized with markers of inflating memory. In the spleen, only antigen-specific CD8 T cells from minigene-immunized mice showed a larger population of upregulated CX3CR1 expression (FIG. 11A), as hypothesized but in the tumour, antigen-specific cells from all groups showed large percentages of CX3CR1hi cells (FIG. 11B). The majority of the antigen-specific cells in the spleen and tumours of all groups were effector memory (FIG. 11C). To investigate if adenoviral immunization alters the tumour microenvironment, the levels of Treg in the tumour and spleen were measured-the levels of Treg in the spleen was slightly elevated in the full-length immunized group, although the levels of Treg in the tumour was not different between groups (FIG. 11D). Likewise, there was no difference in the level of resident memory antigen-specific CD8 T cells in the tumour (FIG. 11E) unlike what was observed in the CT26 tumour model.

### Example 12: CX3CR1hi CD8 T Cells are More Resistant to Oxidative Stress

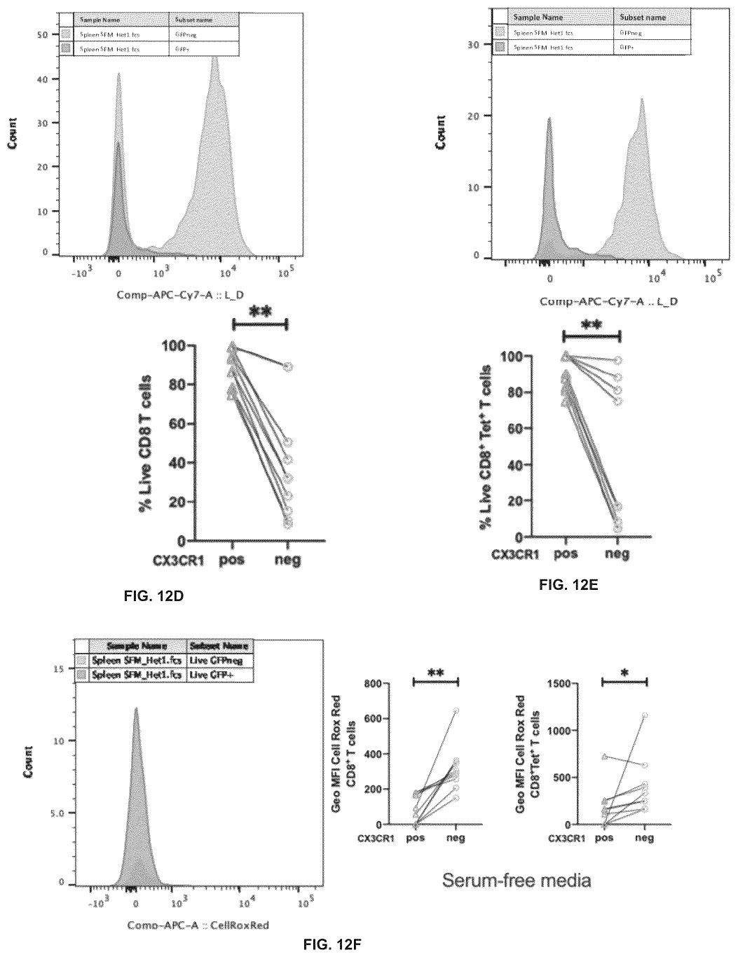

Inflating memory cells upregulate of a number of molecules involved in the anti-apoptotic pathway including Bc1-XL. CX3CR1 expression on human monocytes has been reported to aid cell survival by reducing anti-oxidative stress. We therefore investigated whether CX3CR1 expression conferred a prosurvival effect on inflating memory cells. The levels of intracellular reactive oxygen species (ROS) in CX3CR1+/−gfp splenocytes from Ad-lacZ or MCMV infected mice at day >50 post-infection were detected by CellROX Red assay. We found that in the steady state. CX3CR1hi CD8 T cells contained lower levels of ROS compared to CX3CR1 neg and int CD8 T cell populations (FIGS. 12A and 12C), suggesting CX3CR1hi cells possessed intrinsically lower levels of ROS. Interestingly, CX3CR1hi cells from CX3CR1gfp/gfp mice also possessed lower levels of ROS compared to the CX3CR1 neg subset, indicating that this effect is not solely dependent on CX3CR1 signalling. Also, the levels of reactive oxygen species (ROS) in bulk CX3CR1+ CD8 T cell subset (FIG. 12F, middle) and antigen-specific CX3CR1+ T cells (FIG. 12F, right) remained lower compared to their CX3CR1neg counterpart upon serum starvation thus showing enhanced redox resilience. Additionally, when incubated in serum-free media (i.e. stress), there was a marked survival of the CX3CR1+ population compared to CX3CR1 negative T cells in the bulk (FIG. 12D) and antigen-specific populations (FIG. 12E).

We next determined the percentage of depolarised mitochondria in these subsets by staining peripheral blood lymphocytes from mice persistently infected with MCMV or an adenovector with MitoTracker Green which is used as a marker of mitochondrial mass and MitoTracker DeepRed which stains only polarised, healthy mitochondria. Depolarised mitochondria are positive for MitoTracker Green but not for MitoTracker DeepRed and can be separated from polarised mitochondria by flow cytometry. CX3CR1hi CD8 T cells from wild-type C57BL/6 mice contain a lower percentage of depolarised mitochondria compared to CX3CR1neg CD8

T cells (FIG. 12B). Taken together these results indicate that murine CX3CR1hi CD8 T cells possess a prosurvival advantage over their CX3CR1neg CD8 T cell counterparts, which may promote their long-term persistence and accumulation in the host.

Crucially, cancer is associated with oxidative stress mediated mainly through reactive oxygen species (ROS) generated by malignant cells, granulocytes. TAM and MSDCs in the tumour microenvironment. Therefore, these properties may also protect and preserve their cytotoxic abilities once the cells are inside the tumour.

### Example 13: Immunization with AdHu5-R9F Encoding the Dominant E7 Epitope in HPV Protects Levels Against TC1-HPV E6/E7 Cervical Carcinoma Challenge

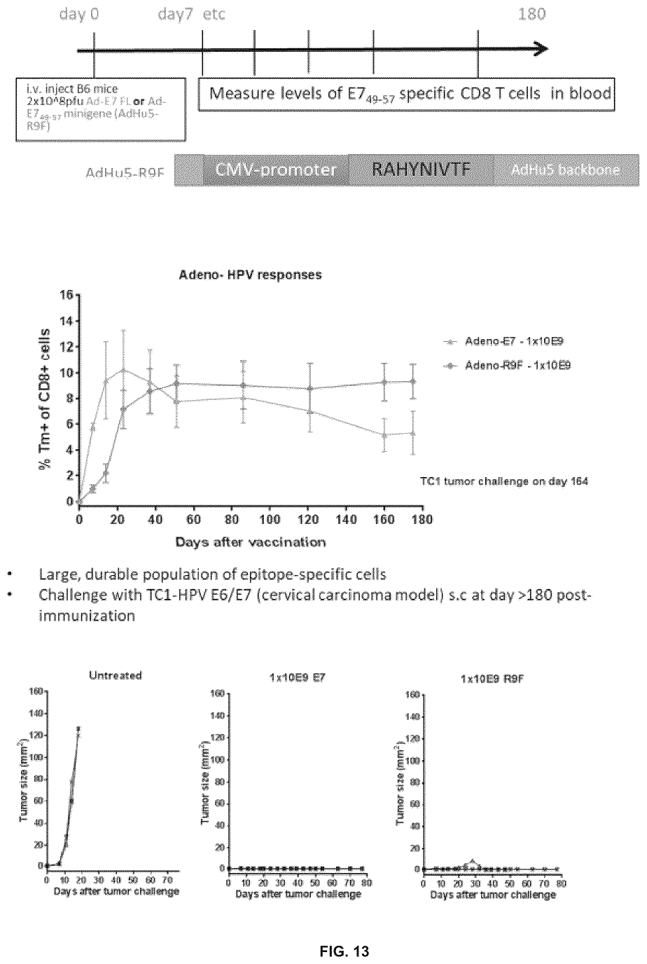

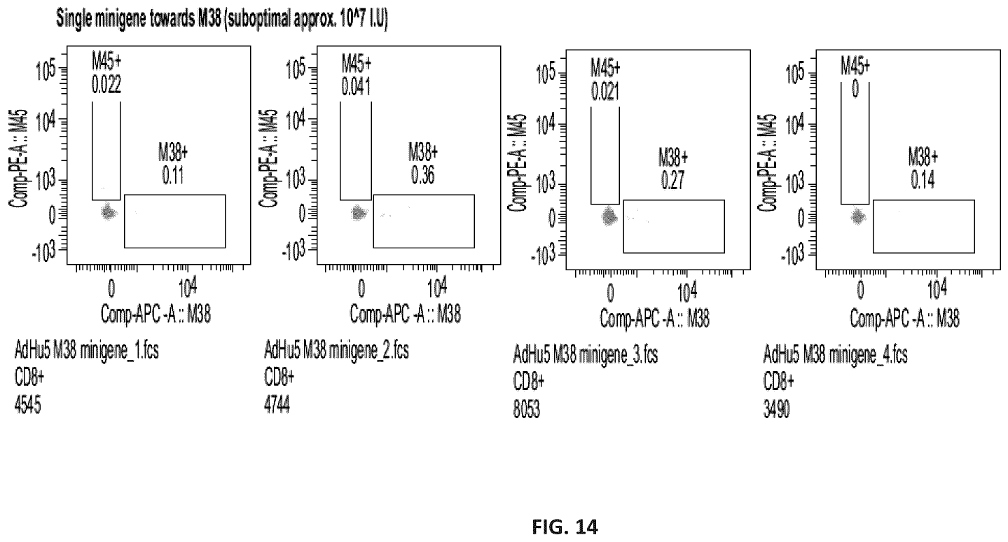

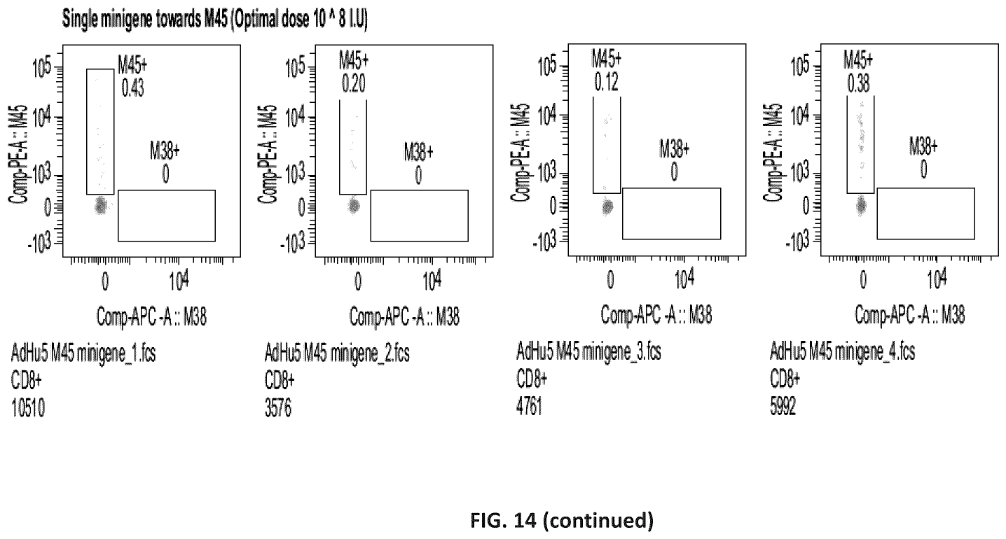

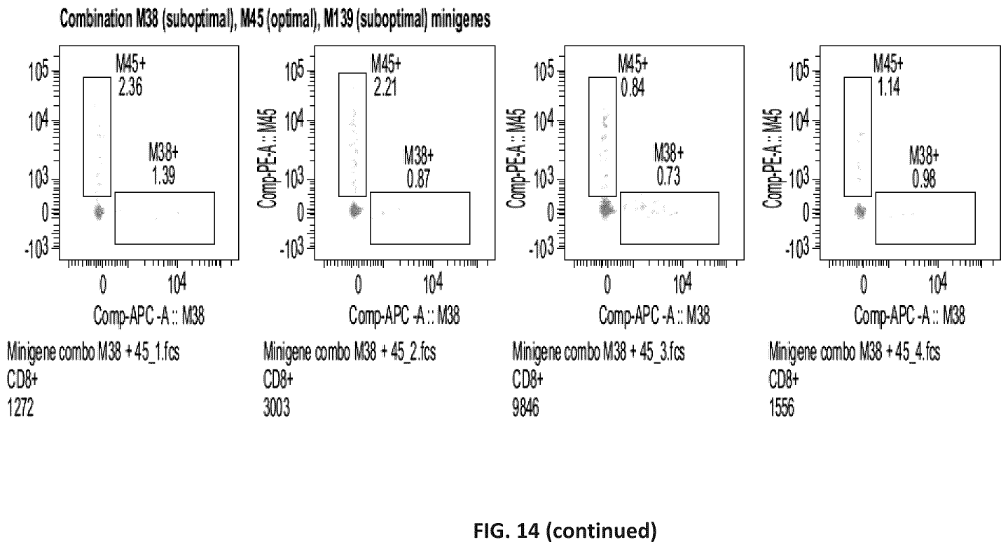

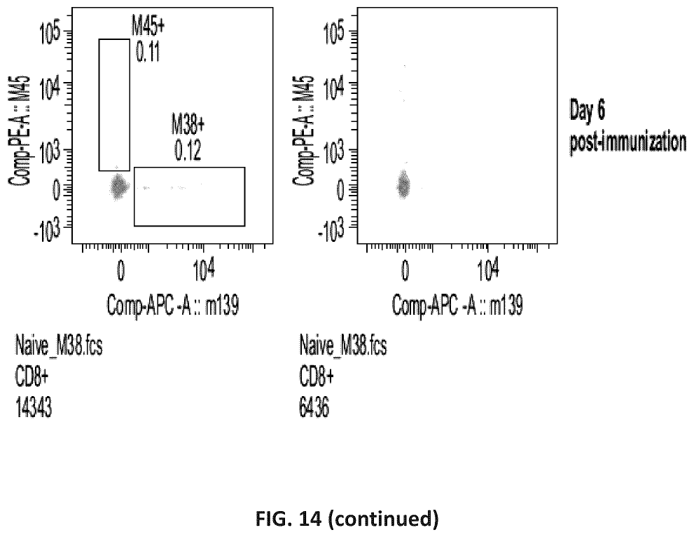

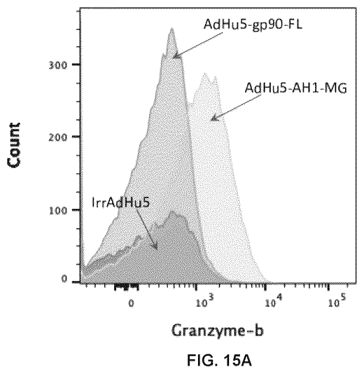

The protection afforded by a minigene construct encoding the dominant CD8 T cell epitope in the E7 protein was compared against that conferred by a similar construct encoding the full-length E7 protein in a prophylactic immunization model. Mice immunized with either construct generated large epitope-specific responses (FIG. 13A) which conferred complete protection upon tumour challenge (FIG. 13B) with no difference observed in the level of protection afford by the whole protein versus the epitope alone.

Example 14: Synergistic Effect After Immunization with a Panel Minigenes Encoding CD8 T Cell Epitopes Against MCMV at a Suboptimal Dose

A panel of 3 minigenes against known MCMV-specific CD8 T cell epitopes, namely M45 (985HGIRNASFI9) 3 SEQ ID NO:10). M38 (316SSPPMFRV325 SEQ ID NO:11) and ml39 (+ 19TWYGFCLL+ 26 SEQ ID NO:12) were constructed. These were injected i.v. into C57BL/6 mice either as individual minigenes or mixed together as a cocktail. The minigene encoding M38 and M139 were injected at a suboptimal dose of 1×10⁷ infectious units (I.U) while the minigene encoding M45 was injected at the optimal dose of 1×10⁸ I.U. The levels of M38-specific cells in the blood at Day 6 post-immunization was measured. Surprisingly, mice that received the combination minigene vaccine containing M38-minigene and m139-minigene vectors at suboptimal doses, plus M45-minigene at optimal dose, developed higher levels of M38-specific T cells compared to the groups injected with only a sub-optimal dose of M38-minigene vector alone. This unexpected result suggests that delivery of a mixture of minigene vectors at suboptimal doses may have additive effect to enhance the magnitude of the antigen-specific T cell over that observed upon immunization with a sub-optimal dose of the single vector alone.

### Example 15: Minigene Immunization Alters the Tumour Environment, Resulting in Higher Levels of Granzyme B

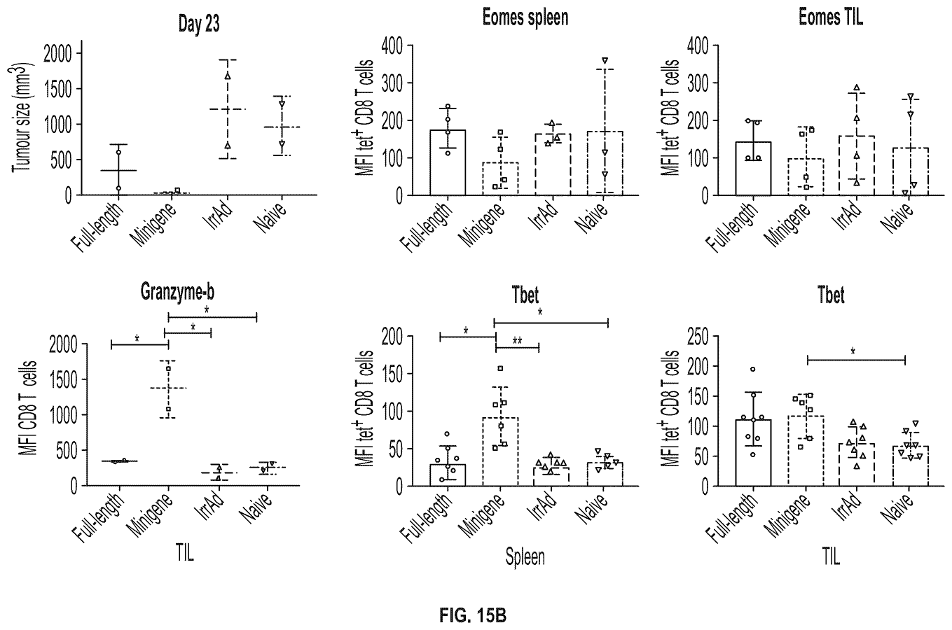

Minigene immunization was performed followed by analysis of the levels of granzyme B. Levels of granzyme B in total CD8+ T cells in the tumours were assessed 23 days post tumour implantation, 16 days post immunization with minigene by intracellular cytokine staining followed by flow cytometry of the single cell suspensions prepared from the tumour, As can be seen in FIG. 15 the level of granzyme B was significantly higher in the CD8+ T cells immunized with the minigene vector compared with when immunization was performed with the full-length epitope vector. The tumour sized was also assessed at 23 days post tumour implantation, FIG. 15 demonstrates that minigene immunization significantly reduced the tumour size compared to controls.

Tetramer+ CD8 T cells were also assessed for the level of transcription factors Eomes and Tbet. Tetramer+ CD8 T cells taken from animals immunized with the minigenes vectors expressed higher levels of Tbet and lower levels of Eomes compared to the tetramer+ cells isolated from the other groups. This is in line with the memory inflation phenotype.

### Example 16: Combination Treatment with Minigene Immunization and Anti-PD-L1 Therapy Enhances Tumour Control

CT26 tumours have been reported to be unresponsive to anti-PD-1 PD-LI monotherapy (Selby etc al., Preclinical Development of Ipilimumab and Nivolumab Combination Immunotherapy: Mouse Tumor Models, In Vitro Functional Studies, and Cynomolgus Macaque Toxicology. PLOS ONE. Public Library of Science: 2016 Sep. 9; 11(9): e0161779-19). However, the present data demonstrates that combination therapy of minigene and anti-PD-L1 results in enhances tumour control and survival.

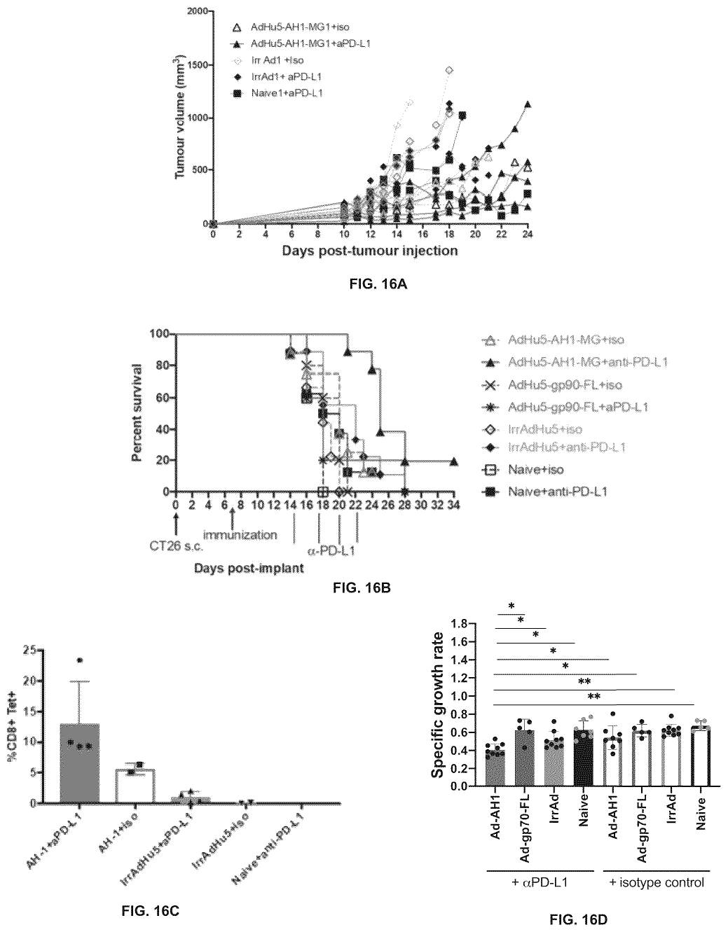

Groups of mice were immunized with the adenoviral vectors as indicated in FIG. 16. 7 days after tumour challenge mice were then administered an anti-PD-L1 or isotype control. FIG. 16A shows that enhanced tumour control (i.e. reduction in tumour size) is observed when the minigene in administered in combination with the anti-PD-L1 therapy. The combination therapy also results in an increased time to humane endpoint of all the treated animals by approx. 33% compared to IrrAdHu5 immunized untreated subjects. Survival curves of all groups of mice are shown in FIG. 16B. The % of GP70₄₂₃₋₄₃₁ Tet+ cells in circulation 15 days after immunization (22 days post-tumour challenge) was assessed. Combination therapy increased the levels of tetramer+ cells in circulation compared to minigene-alone treatments (FIG. 16C) and significantly reduced the growth rate of the tumors (FIG. 16D).

### Example 17. Analysis of IFNγ Production in Tumour and Spleen Derived Cells

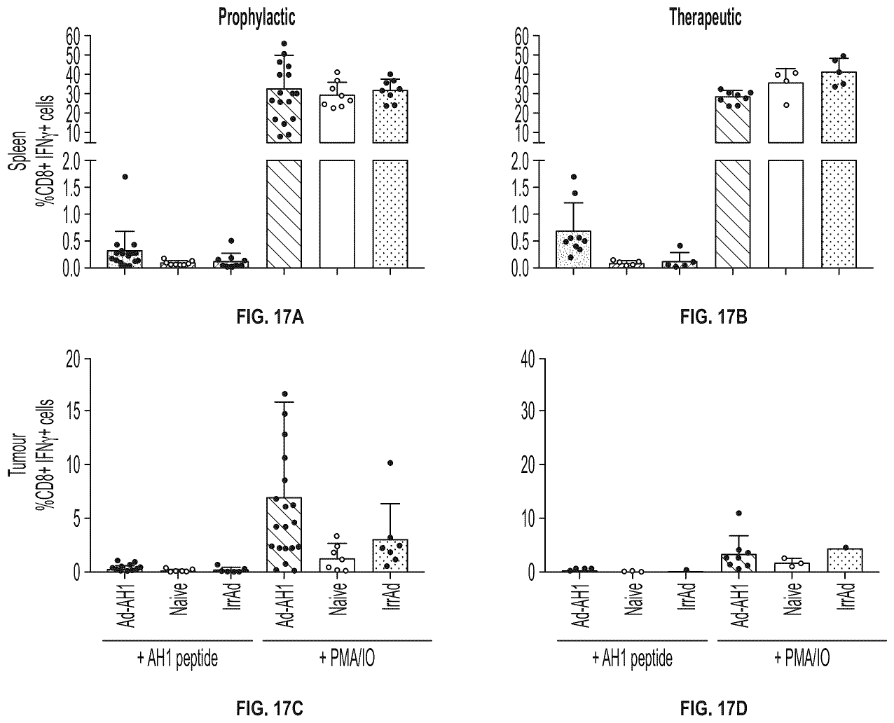

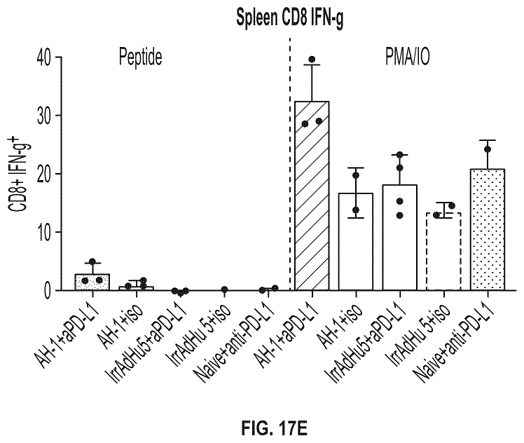

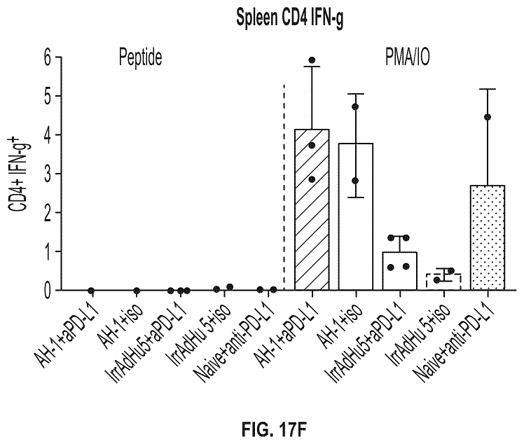

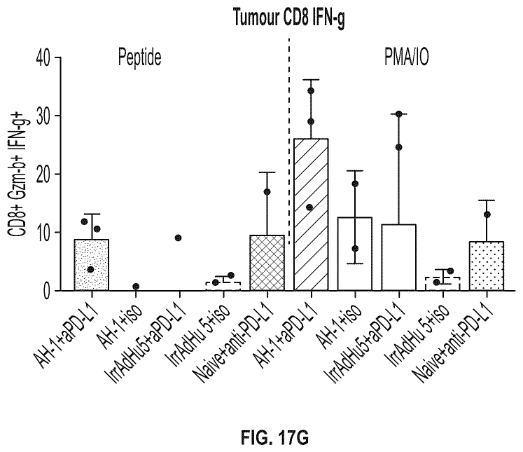

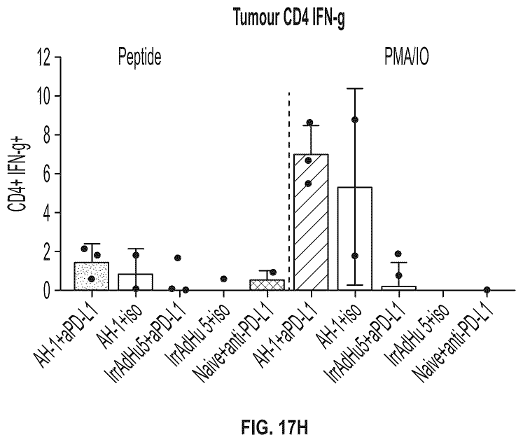

Spleen- and tumour-derived single cells were obtained from mice immunized both prophylactically and therapeutically and were stimulated ex-vivo with AH1-peptide (4 μg/ml) or PMA-Ionomycin (IO) for 7 hours and then stained for intracellular cytokine production of IFNγ. IFNγ-secreting cells were detected and elevated only in the spleens of prophylactic (FIG. 17A) or therapeutic (FIG. 17B) immunized groups, with low/no IFNγ-secreting cells detected in the tumour (FIG. 17C and FIG. 17D). Therapeutic vaccination combined with anti-PD-L1 experiments were stimulated ex vivo with AH1-peptide (4 μg/ml) or PMA-Ionomycin (IO) for 7 hours and then stained for intracellular cytokine production of IFNγ. IFNγ-secreting CD8 T cells in both spleen and tumour were increase in the samples treated with the combination therapy compared to minigene-alone treatments (FIG. 17E and FIG. 17G). In the CD4 T cell compartment. IFNγ-secreting CD4 T cells could be detected in the tumors (FIG. 17H) of vaccinated combined with anti-PD-L1 group but not the spleen (FIG. 17F).

### Example 18. Immunization with a Combination of Two AdHu-5 Minigenes (MG) Encoding Two Different Tumour Antigens Confers Enhanced Survival over Immunization with Single in a Therapeutic Immunization Model

Mice were s.c. implanted with CT26 tumour cells (5×10⁵ cells/mouse). 6 days later mice were vaccinated with single minigene vaccines each encoding a different CT26 tumour antigen. AdHu5-AH1-MG or AdHu5-e2F8-27merMG at 1×10⁸ IU. or both minigene vaccines together (Combo, both at 1×10⁸IU). Half of each group was treated with the checkpoint inhibitor anti-PD-1 at 12, 16 and 19 days post-implantation and half the group were treated with an isotype control. Tumour growth was monitored until it approached 1.3 cm³.

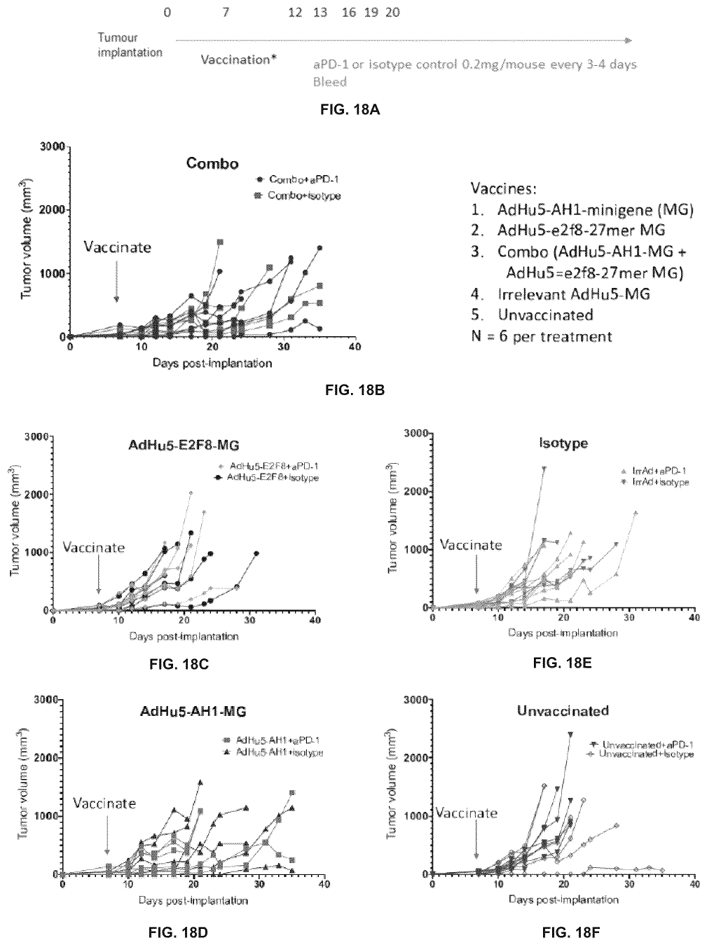

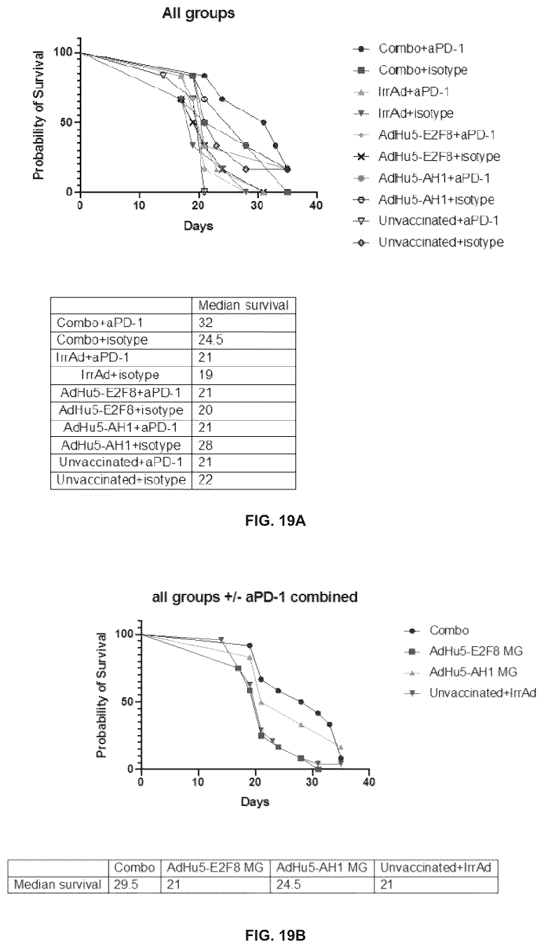

FIG. 18B-F show vaccination with combination vaccines (Combo) slowed tumour growth compared to the negative controls (unvaccinated or vaccinated with AdHu5-MG encoding an irrelevant antigen). FIG. 19A demonstrates combination vaccine treatment plus anti-PD-1 enhanced survival over the negative control while treatment with combination vaccines in general increased the median survival compared to negative controls or groups vaccinated with a single minigene vaccine only as shown in FIG. 19B.

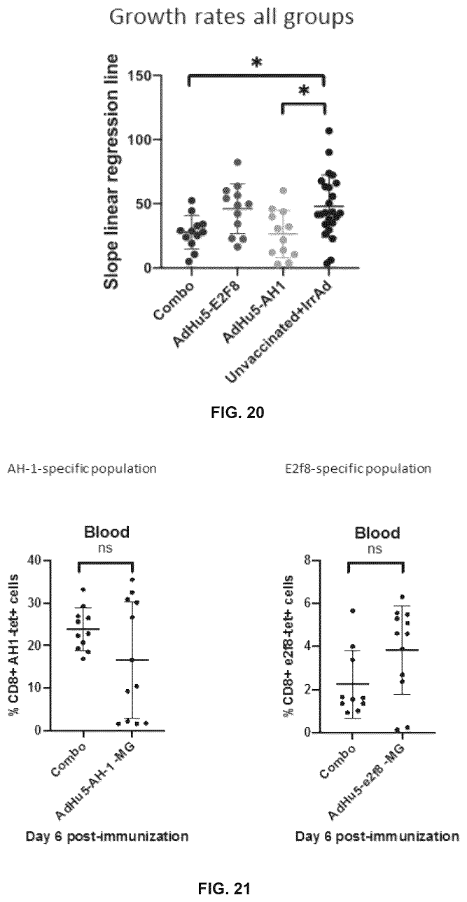

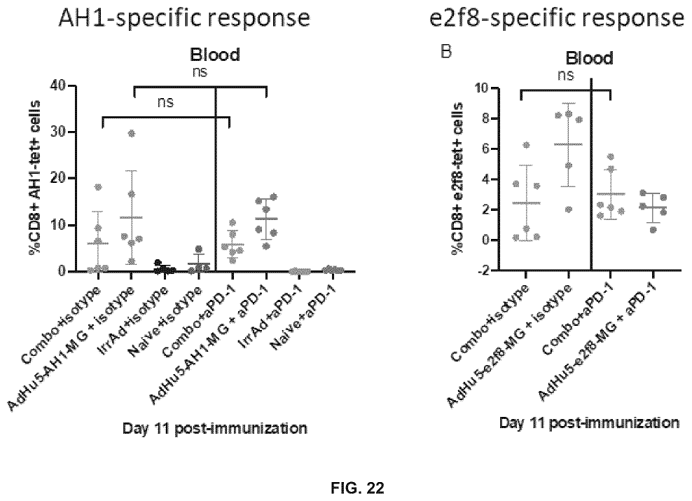

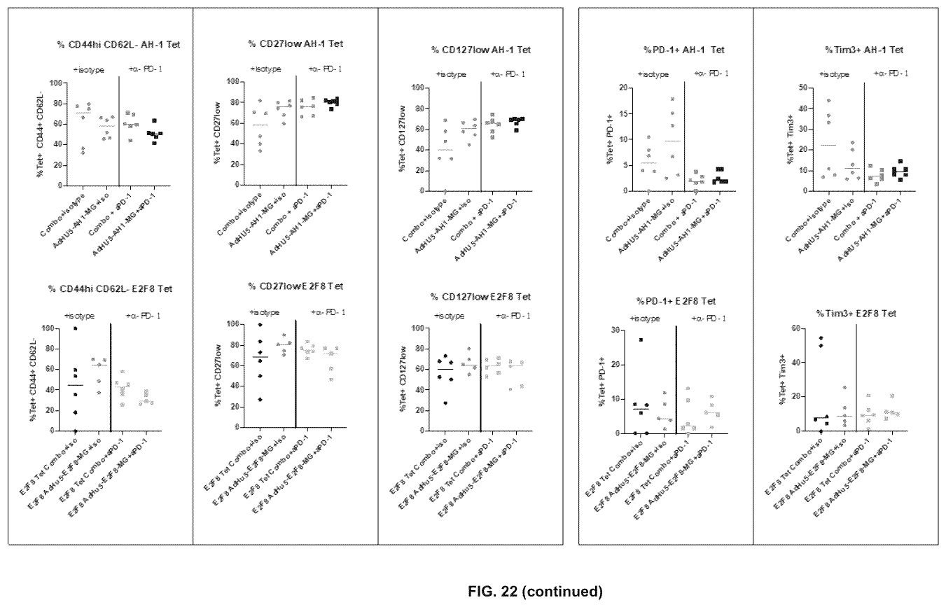

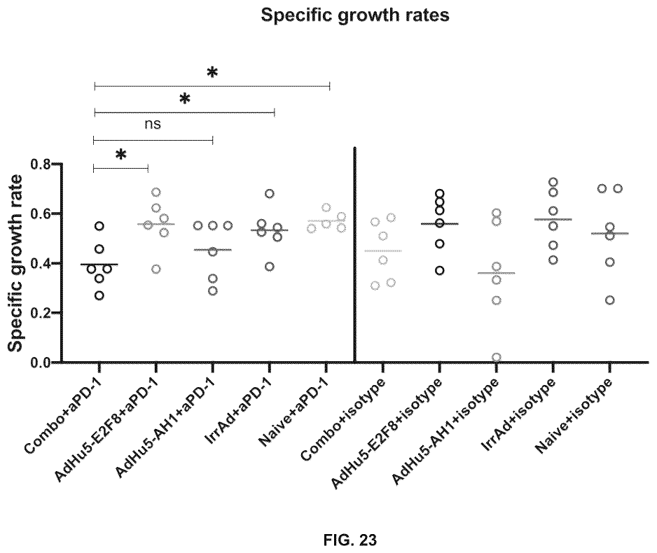

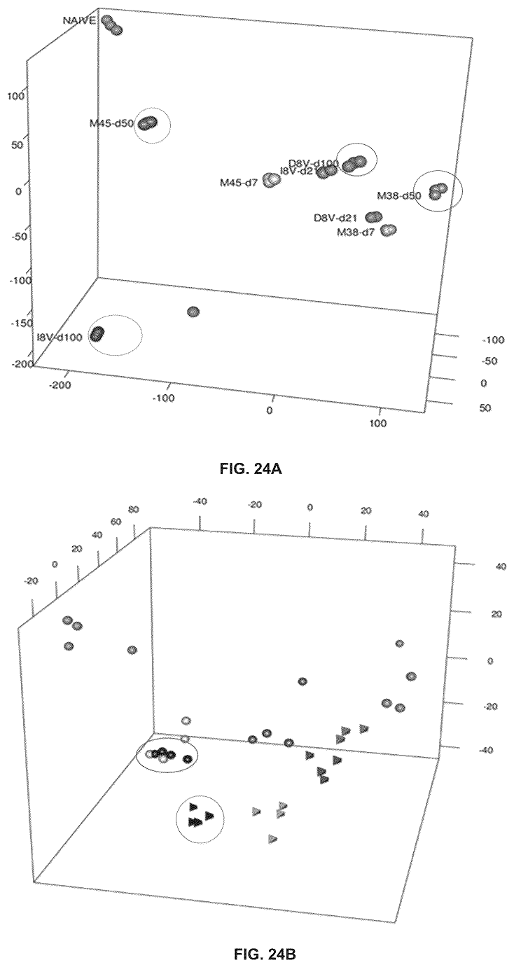

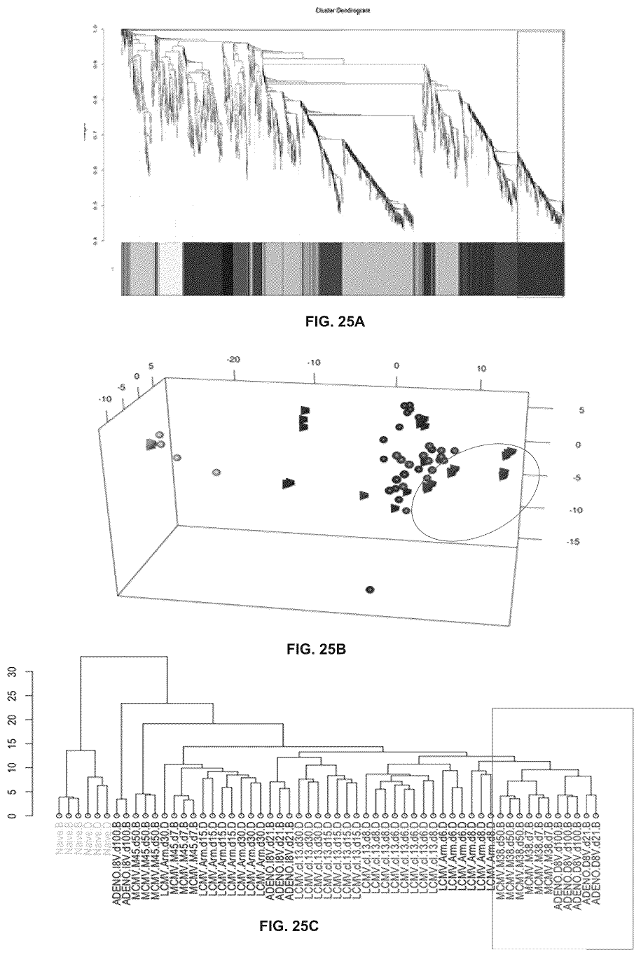

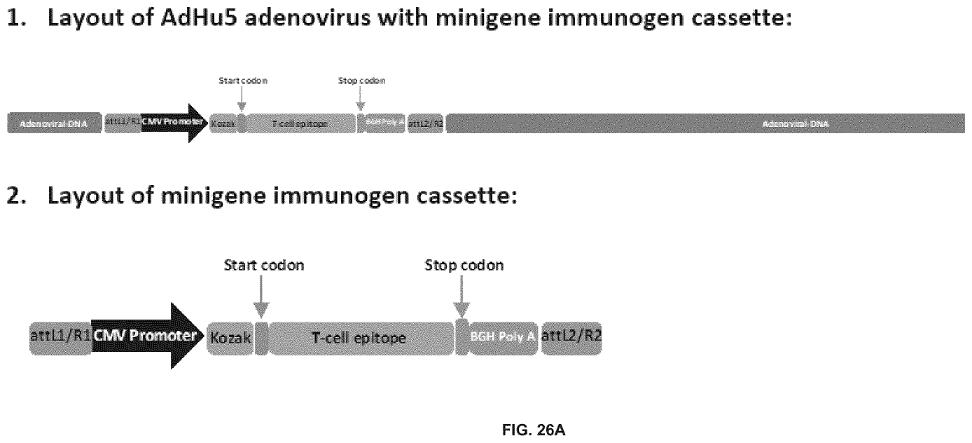

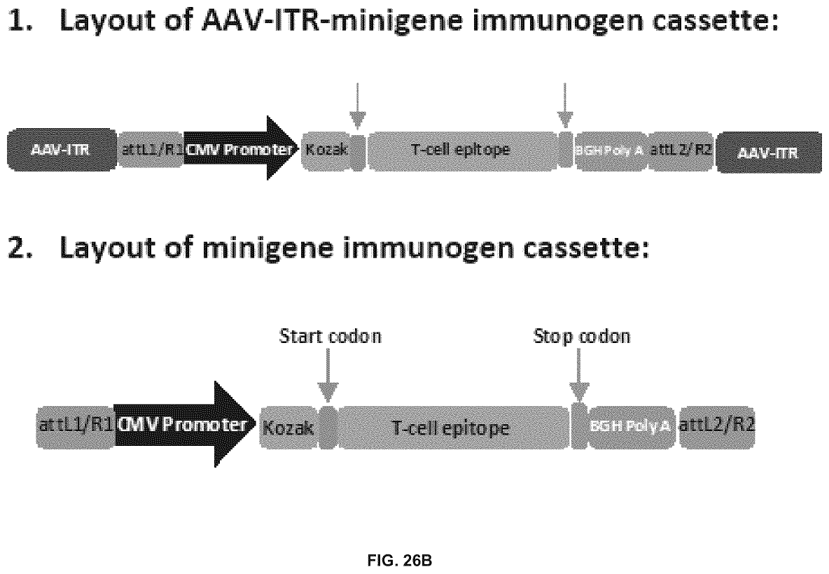

The growth rate of the tumours were determined by simple linear regression analysis of the tumour sizes over time to calculate the slope of the curve (steeper=higher growth rate) (FIG. 20). Alternatively, the same data was used to calculate the growth rate as specific growth rates (FIG. 23). The values of individual mice according to vaccination type are shown. Combination vaccination significantly slowed down tumour growth compared to the negative control groups (FIG. 20 and FIG. 23). Blood was sampled 6 days post-vaccination and stained with surface markers against CD8 and tetramers specific for AH-1 or e2f8 antigen (FIG. 21). The % Tet+ in the live CD8 T cell compartment is shown. Vaccination with combination vaccines increases the magnitude of the AH-1 tet+ population compared to the group vaccinated AdHu5-AH-1 MG only (FIG. 21). FIGS. 22 and 23 demonstrate that simultaneous i.v. immunization with two minigene constructs/vaccines (combo) induces both antigen-specific populations at similar magnitudes and phenotype to single vaccine and act to control tumour growth.

### Methods

### Animals

Mouse experiments were performed according to UK Home Office regulations (project licence numbers PBA43A2E4 and PPL 30/3293) and approved by the local ethical review board at the University of Oxford. Male and female mice were maintained in Specific Pathogen Free (SPF) conditions in individually ventilated cages and fed normal chow diet. Adult HHD mice transgenic for HLA-A2 were bred at the university's BSL2 facility and kindly provided by Vincenzo Cerundolo (HIU, University of Oxford, Oxford). Balbc mice aged 6-8 weeks were obtained from Charles River (Margate, UK).

### Adenoviral Vectors

For the NY-ESO-1 studies, the full-length NY-ESO-1 gene or the dominant CD8 T cell epitope SLWTQC was cloned into the AdHu5 vector backbone. For the CT-26 studies, the full-length Murine Leukemia virus gene gp⁹⁰ or the dominant CD8 T cells epitope SPSYVYHQF (SEQ ID NO:1) was inserted as above to generate the constructs AdHu5-FL and AdHu5-AH1-MG. The constructs were scaled up, purified and quantitated by the Viral Vector Core Facility (Oxford, UK) in 293A cells with purification by Caesium Chloride centrifugation and stocks were stored at −80° C. in PBS. A second construct AdHu5-e2f8-27 MG encoding an immunogenic mutation from CT26 tumour containing a predicted CD8 T cell epitope, VILPQAPSGPSYATYLQPAQAQMLTPP (SEQ ID NO:4), was generated, scaled up in 293A cells and purified by membrane purification (Sartorious).

For HPV 16 E7 studies, the full length HPV16 E7 gene or the dominant CD8 T cell epitope RAHYNIVTF (SEQ ID NO:7) was cloned into the AdHu5 vector backbone. Control vectors comprised of the CD8 T cell epitope ICPMYARV (SEQ ID NO:8) from the bacterial enzyme β-galactosidase inserted into the AdHu5 vector backbone,

Mouse Immunizations and Tumour Challenge and Treatment with Anti-PD-L1 Antibody

Mice were immunized intravenously by tail vein injection with 1×10⁷⁻⁹ infectious units (IU) of virus as indicated. The HHD-sarcoma cell line transgenic for NY-ESO-1 or CT26 colorectal cancer or TC-1 (HPV 16 E7 expressing) cell lines, were injected s.c. in the flank at between 0.1-1×10⁶ cells/200 μl. Mycoplasma testing was performed on the cell lines prior to injection and only mycoplasma negative cells were used.

Mice were monitored post tumour challenge and when palpable, the tumour diameters were measured every 1-2 days using digital callipers and the volume calculated using the modified ellipses formula, Volume=(width²×length/2, to determine the rate of tumour growth.

For therapeutic challenge studies, mice were first implanted with tumour cells s.c. in the flank—6-7 days later the animals were immunized intravenously via the tail vein with the relevant adenoviral vectors at 1×10⁷⁻⁹ IU and the tumours measured as before. In some experiments, mice were treated with 0.2 mg of either anti-mouse PD-L1 (clone 10F.9G2, Biolegend) or isotype control by i.v. injection at days 14,17,20 and 22 post-tumour implantation.

Lymphocyte Isolation from Blood and Tissues

Blood, spleen and tumour samples were processed using enzymatic and mechanical digestion to obtain lymphocyte populations with high viability. Tumours were excised and then digested with collagenase and DNAse for 45 mins at 37° C. The digested tumours were passed through a 100 μm cell sieve, then washed with complete RPMI and pelleted by centrifugation at 1500 rpm for 5 mins. The cell pellet was resuspended and then passed through a 40 μm cell sieve, before being washed and pelleted as before. The isolated tumour cells were then resuspended and counted.

### Detection and Analysis of Tumour and Vaccine-Specific T Cells

Details of tetramers and pentamers used to detect virus and vaccine-specific T cells are shown in Table 2.

TABLE 2

| | T | | Abbre- | |
|---|---|---|---|---|
| HLA | arget- | Amino acid | viation/ | |
| type | epitope | sequence | SEQ ID NO | Source |
| Human | | | | |
| A*0201 | NY-ESO-1₁₅₇₋₁₆₅ | SLLMWITQC | NY-ESO-1 | NIH |
| | | | SEQ ID | Tetramer |
| | | | NO: 2 | Facility |
| Mouse | | | | |
| H-2Ld | MuLV | SPSYVYHQF | AH1 | NIH |
| | envgp70₄₂₃₋₄₃₁ | | SEQ ID | Tetramer |
| | | | NO: 1 | Facility |
| H-2Dd | MuLV gp90₁₄₇₋₁₄₈ | GGPESFYCASW | GSW11 | NIH |
| | | | SEQ ID | Tetramer |
| | | | NO: 3 | Facility |
| H-2Kd | Mtch1₃₆₁₋₃₆₉ | KYLSVQSQL | Mtch1 | Immudex |
| | | | (9 mer) | |
| | | | SEQ ID | |
| | | | NO: 6 | |
| H-2Kd | MtCh1₃₆₁₋₃₇₀ | KYLSVQSQLF | MTCH1 | Immudex |
| | | | (10 mer) | |
| | | | SEQ ID | |
| | | | NO: 5 | |
| H-2Dd | E2f8₅₁₆₋₅₂₄ | SGPSYATYL | e2f8 | Immudex |
| | | | SEQ ID | |
| | | | NO: 38 | |
| H-2Kb | βgal₄₉₇₋₅₀₄ | ICPMYARV | I8V | NIH |
| | | | SEQ ID | Tetramer |
| | | | NO: 8 | Facility |
| H-2Ld | MCMV- | YPHFMPTNL | pp89 | NIH |
| | m123/pp89₁₆₈₋₁₇₆ | | SEQ ID | Tetramer |
| | | | NO: 9 | Facility |
| H-2Db | MCMV-M45₉₈₅₋₉₉₃ | HGIRNASFI | M45 | NIH |
| | | | SEQ ID | Tetramer |
| | | | NO: 10 | Facility |
| H-2Kb | MCMV-M38₃₁₆₋₃₂₄ | SSPPMFRV | M38 | NIH |
| | | | SEQ ID | Tetramer |
| | | | NO: 11 | Facility |
| H-2Kb | MCMV-m139₄₁₉₋₄₂₆ | TWYGFCLL | m139 | NIH |
| | | | SEQ ID | Tetramer |
| | | | NO: 12 | Facility |
| H-2Db | HPV16 E7₄₉₋₅₇ | RAHYNIVTF | E7₄₉₋₅₇ | NIH |
| | | | SEQ ID | Tetramer |
| | | | NO: 7 | Facility |

The reagents listed in Table 2 were synthesized as monomers and tetramerized by addition of streptavidin-PE (BD Bioscience) or streptavidin-APC (Invitrogen, Paisley, UK). Peptides for construction of the monomers was obtained from Proimmune (Oxford, UK). Aliquots of approx. 50 μl of whole blood were stained using 50 μl of a solution containing tetrameric class I peptide complexes at 37° C. for 20 min followed by staining with mAbs and fixable NIR LIVE/DEAD stain.

### Antibody Staining

Single cell suspensions were blocked with a FcR-blocking reagent (CD16/CD32, eBiosciences) (20 minutes at 4° C.) to prevent nonspecific antibody binding. Subsequently, cells were immunostained with tetramer (as described above) and various fluorochrome-conjugated antibodies (20 minutes at 4° C.). In all antibody panels fixable viability dye (LIVE/DEAD™ near-IR dye (Invitrogen)) was added to exclude dead cells from analysis. The following antibodies were used for flow cytometry at a concentration of 1:100 with exceptions marked in the list: CD4-AF700 (RMA4-4, Biolegend), CD8 (53-6.7 eBiosciences or Biolegend), CD11a/CD18/LFA-1 (H155-78, Biolegend), CD25 (PC61.5, eBiosciences), CD27 (LF.3A10, Biolegend), CD44 (IM7, eBiosciences), CD62L (MEL-14, Biolegend), CD69 (H1.2F3, Biolegend, 1/200), CD95/Fas (Jo2 BD), CD103 (2E7, Biolegend, 1/200), CD127 (SB/199, Biolegend), CD279/PD-1 (RMP1-30, Biolegend), CX3CR1 (SA011F11, Biolegend), FoxP3 (FJK-16s, eBiosciences), IFN-γ (XMG1.2, eBiosciences), IL-2 (JES6-5H4, eBiosciences), KLRG1 (2F1, abcam), TNF-α (MP6-XT22, eBiosciences). Prior to fixation and permeabilization of single cell samples (using the FoxP3/Transcription Factor staining buffer set, Invitrogen) required for intracellular staining, extracellular staining was performed. For intracellular cytokine staining, tumour-or spleen-derived single cells were stimulated ex vivo with peptide (4 μg/ml) alongside positive (PMA at 2 μg/ml and IO at 4.4 μg/ml) and negative (medium only) controls for 2,5 hours after which cells were incubated with GolgiPlug (BD, 1 μl/ml) for 4,5 hours at 37° C. Antibodies used are listed in the table below. These were used at 1:100 dilution except where indicated.

TABLE 3

| | | | Catalogue | |
|---|---|---|---|---|
| Antibodies | Supplier | Clone | number | Dilution |
| Anti-human/mouse | eBioscience | IM7 | 1929433 | |
| CD44 FITC | | | | |
| Anti-human/mouse | Biolegend | GB11 | B243514 | |
| Granzyme-b PB | | | | |
| Anti-human/mouse T-bet | eBioscience | eBio4B10 | E12135-1631 | |
| APC | | | | |
| Anti-mouse CD103 | Biolegend | 2E7 | B227281 | 1/200 |
| Pacific Blue/BV421 | | | | |
| Anti-mouse | Biolegend | H155-78 | 141011 | |
| CD11a/CD18/LFA-1 | | | | |
| Anti-mouse CD127 APC | Biolegend | SB/199 | 121122 | |
| Anti-mouse CD223 | Biolegend | C9B7W | B261545 | |
| (LAG-3) PerCP Cy5.5 | | | | |
| Anti-mouse CD25 AF700 | Biolegend | PC61 | B102024 | |
| Anti-mouse CD25(IL-2R | BD Pharmingen | / | M056210 | |
| a-chain) FITC | | | | |
| Anti-mouse CD27 | Biolegend | LF.3A10 | 124214 | |
| PerCP/Cy5.5 | | | | |
| Anti-mouse CD279 | Biolegend | RMP1-30 | B228182 | |
| (PD-1) PE-Cy7 | | | | |
| Anti-mouse CD4 AF700 | eBioscience | GK1.5 | 4313129 | |
| Anti-mouse CD4 BV650 | Biolegend | GK1.5 | B282965 | |
| Anti-mouse CD4 Pacific | eBioscience | RM4-5 | E08484-1634 | |
| Blue | | | | |
| Anti-mouse CD44 | Biolegend | IM7 | B230511 | |
| BV605 | | | | |
| Anti-mouse CD62L | Biolegend | MEL-14 | B268247 | |
| AF700 | | | | |
| Anti-mouse CD62L PE | eBioscience | MEL-14 | E07577-1631 | |
| Cy7 | | | | |
| Anti-mouse CD69 | Biolegend | H1.2F3 | B237998 | |
| BV605 | | | | |
| Anti-mouse CD69 PerCP | Biolegend | H1.2F3 | B277659 | 1/200 |
| Cy5.5 | | | | |
| Anti-mouse CD8a AF700 | eBioscience | 53-6.7 | E08952-1632 | |
| Anti-mouse CD8a APC | Biolegend | 53-6.7 | B244174 | |
| Anti-mouse CD8a | Biolegend | 53-6.7 | B253266 | |
| BV650 | | | | |
| Anti-mouse CD8a FITC | Biolegend | 53-6.7 | B277418 | |
| Anti-mouse CD8a | eBioscience | 53-6.7 | E08488-1632 | |
| PB/efluor450 | | | | |
| Anti-mouse CD8a PerCP | Biolegend | 53-6.7 | B249622 | |
| Cy5.5 | | | | |
| Anti-mouse CD95/Fas | BD | Jo2 | 554259 | |
| Anti-mouse CX3CR1 | Biolegend | SA011F11 | B262849 | |
| BV421 | | | | |
| Anti-mouse Fas FITC | Pharmingen | / | M076296 | |
| Anti-mouse IFN-γ PE | Invitrogen | XMG1.2 | 2028218 | |
| Anti-mouse IL-2 APC | Biolegend | JES6-5H4 | B248052 | |
| Anti-mouse KLRG1 FITC | Abcam | 2F1 | ab24867 | |
| Anti-mouse Tim-3 APC | Biolegend | B8.2C12 | B228873 | |
| Anti-mouse Tim-3 | Biolegend | RMT3-23 | B262042 | |
| BV605 | | | | |
| Anti-mouse Tim-3 PerCP | Biolegend | RMT3-23 | B224464 | |
| Cy5.5 | | | | |
| Anti-mouse TNFa FITC | Invitrogen | MP6-XT22 | 1927452 | |
| Anti-mouse/human | Biolegend | IM7 | B250780 | |
| CD44 PI/PE-Texas Red | | | | |
| Anti-mouse/Rat FoxP3 | Invitrogen | FJK-16S | 4344418 | |
| PE-Cy7 | | | | |
| Eomes PerCP Cy5.5 | eBioscience (San | Dan11mag | E12115-1631 | |
| | Diego, CA) | | | |
| L/D (near IR fluorescent | Invitrogen | / | 1937144 | |
| reactive dye) APC Cy7 | | | | |

### Flow Cytometry

All immunostained samples were analysed by flow cytometry using a BD LSR II Flow Cytometer. Data analysis was conducted using the software FlowJo v10. Cells were gated on lymphocytes, single cells, live cells, and subsequent relevant markers for analysis.

### CellROX Red Assay

Single cell splenocytes were prepared from CX3CR1gfp/+ or gfp/gfp mice infected >50 previously with MCMV or Ad-lacZ. The splenocytes were plated out into 96-well plates and cultured in complete media (RPMI+10% FCS) for 48 hours. The cells were spun down and washed with 200 μl sterile DPBS (Life Technologies). The cells were then treated with either serum-free RPMI or RPM+10% FCS (added at 40 μl per well). These were incubated for 1-1.5 hours at 37 C. CellROX red reagent (Life Technologies) was diluted 1:50 with serum-free media and then 4 μl of diluted reagent was added to each well and incubated for 40 mins at 37 C. The cells were then stained with appropriate surface antibodies (appropriate tetramer-PE, CD8-eFluor 450, CD62L-AlexaFluor 700, CD44-PerCP-Cy5.5 and Fixable Live Dead marker) for 20 mins at 37 C. Cells were washed with PBS and then resuspended in PBS and analysed on an LSRII and the geometric mean of CellROX red on live CD8 T cells calculated on FlowJo software.

### Mito Tracker Assay

PBL from C57BL/6 mice infected >100 days previously with MCMV or an AdHu5 recombinant adenovector (Ad-18V) were stained with anti-mouse CD8, anti-mouse CX3CR1, LiveDead nearIR Fixable Marker. Staining with 12.5 nm MitoTracker Green and 12.5 nm MitoTracker DeepRed (Fisher Scientific) for 30 min at 37° C. was carried out prior to surface staining and then analysed on an LSRII and the data calculated on FlowJo.

Statistical analysis

Descriptive statistics (percent means, standard deviations, counts) were calculated using GraphPad PRISM (Graphpad software, Inc., La Jolla, CA). P-values for comparison of means was determined by T test, one-way and two-way ANOVA and corrected using Holm-Sidak for multiple comparisons. Statistical significance was defined as p<0.05.

### Method for Recombinant AAV-Minigene Production

Recombinant AAV encoding a minigene of interest will be generated by transfecting HEK 293 cells with three plasmids: (1) AAV-ITR plasmid containing the minigene of interest [AAV-ITR-minigene], (2) an adenovirus helper plasmid that encodes the E2A, E4 and VA adenoviral proteins that are required for AAV replication and (3) a helper plasmid encoding the rep and cap genes of AAV, required for packaging the AAV-ITR-minigene within the AAV viral particles.

### Vector Sequences:

The following provides exemplary sequences that may be used in the vector of the present invention.
SEQ ID NO:13 AdHu5 adenovirus nucleotide sequence 5′ to the minigene immunogen cassette:
SEQ ID NO:14 AdHu5 adenovirus nucleotide sequence 3′ to the minigene immunogen cassette:

### Minigene Immunogen Cassette:

SEQ ID NO:15 Minigene immunogen cassette nucleotide sequence 5′ to the T cell epitope
   Minigene immunogen cassette nucleotide sequence 5′ to the T cell epitope sequence
SEQ ID NO:16 attR1 sequence
SEQ ID NO:17 attL1 sequence
SEQ ID NO:18 CMV promoter sequence
SEQ ID NO:19 Kozak sequence
SEQ ID NO:20 Start codon
   T cell epitopes:
SEQ ID NO:2 NY-ESO-1 epitope
SEQ ID NO:21 Homo sapiens codon optimized NY-ESO-1 epitope nucleotide sequence
SEQ ID NO:1 AH1 epitope
SEQ ID NO:22 Mus Musculus codon optimized AH1 epitope nucleotide sequence
SEQ ID NO:3 GSW11 epitope
SEQ ID NO:23 Mus Musculus codon optimized GSW11 epitope nucleotide sequence
SEQ ID NO:4 e2f8 epitope
SEQ ID NO:24 Mus Musculus codon optimized e2f8 epitope nucleotide sequence
SEQ ID NO:5 Mtch1-10mer epitope
SEQ ID NO:25 Mus Musculus codon optimized Mtch1-10mer epitope nucleotide sequence
SEQ ID NO:6 Mtch 1-9mer epitope
SEQ ID NO:26 Mus Musculus codon optimized Mtch1-9mer epitope nucleotide sequence
SEQ ID NO:8 18V epitope
SEQ ID NO:27 Mus Musculus codon optimized I8V epitope nucleotide sequence
SEQ ID NO:9 pp89 epitope
SEQ ID NO:28 Mus Musculus codon optimized pp89 epitope nucleotide sequence
SEQ ID NO:10 M45 epitope
SEQ ID NO: 29 Mus Musculus codon optimized M45 epitope nucleotide sequence
SEQ ID NO:11 M38 epitope
SEQ ID NO:30 Mus Musculus codon optimized M38 epitope nucleotide sequence
SEQ ID NO:12 m139 epitope
SEQ ID NO:31 Mus Musculus codon optimized m139 epitope nucleotide sequence
SEQ ID NO:7 HPV16 E749-57 epitope
SEQ ID NO:32 Mus Musculus codon optimized HPV16 E749-57 epitope nucleotide sequence
SEQ ID NO:33 Minigene immunogen cassette nucleotide sequence 3′ to the T cell epitope sequence

### Minigene Immunogen Cassette Nucleotide Sequence 3′ to the T Cell Epitope Sequence:

Stop codon
SEQ ID NO: 34 BGH poly A sequence
SEQ ID NO:35 attL2 sequence
SEQ ID NO:36 attR2 sequence

The minigene immunogen cassette described above may be used with an AAV vector. For example an AAV vector comprising inverted terminal repeats may be used. An example sequence is provided below.
SEQ ID NO:38 AAV nucleotide sequence 5′ to the minigene immunogen cassette

### Descriptions for AAV Nucleotide Sequence 5′ to the Minigene Immunogen Cassette:

SEQ ID NO:39 5′ ITR nucleotide sequence
SEQ ID NO:40 Extra sequences 5′ to the minigene immunogen cassette
SEQ ID NO:41 AAV adenovirus nucleotide sequence 3′ to the minigene immunogen cassette

### Descriptions for AAV Adenovirus Nucleotide Sequence 3′ to the Minigene Immunogen Cassette:

SEQ ID NO:42 3′ ITR nucleotide sequence
SEQ ID NO:43 5′ Extra sequences 3′ to the minigene immunogen cassette

What is claimed is:

1. A method of inducing an inflating memory CD8+ T cell response comprising:
administering a therapeutically effective amount of a pharmaceutical composition to a subject with a tumor, wherein the pharmaceutical composition comprises an adenoviral vector comprising a nucleotide sequence encoding a polypeptide comprising a single cancer-specific CD8+ T cell epitope, and wherein the adenoviral vector does not comprise a nucleic acid molecule encoding a cancer-specific T cell epitope other than the single cancer-specific CD8+ T cell epitope.

2. The method of claim 1, wherein the adenoviral vector persists in a cell of the subject for at least 28 days.

3. The method of claim 1, wherein the single cancer-specific CD8+ T cell epitope is not presented by a class II MHC molecule and does not activate a CD4+ T cell.

4. The method of claim 1, wherein the polypeptide comprising the single cancer-specific CD8+ T cell epitope is not processed by antigen presenting cells of the subject after administration.

5. The method of claim 1, wherein the nucleotide sequence encoding the single cancer-specific CD8+ T cell epitope is from 21 and 45 bases in length and the single cancer-specific CD8+ T cell epitope is from 7 to 15 amino acids in length.

6. The method of claim 1, wherein the single cancer-specific CD8+ T cell epitope is from a protein that is overexpressed in a tumor cell or from a protein expressed in a tumor cell that is mutated.

7. The method of claim 6, wherein growth of the tumor is inhibited in the subject to a greater extent compared to administration of a corresponding pharmaceutical composition comprising a corresponding adenoviral vector comprising (i) a nucleotide sequence encoding a full-length version of the protein that is expressed in the tumor cell, (ii) a nucleotide sequence containing two or more copies of the sequence encoding the cancer specific CD8+ T cell epitope, or (iii) a nucleotide sequence comprising a sequence encoding the cancer specific CD8+ T cell epitope and a sequence encoding a different cancer specific T cell epitope.

8. The method of claim 1, wherein the tumor is a cancer selected from the group consisting of colorectal cancer, prostate cancer, esophageal cancer, liver cancer, renal cancer, lung cancer, breast cancer, pancreatic cancer, brain cancer, hepatocellular cancer, gastric cancer, cervical cancer, ovarian cancer, thyroid cancer, melanoma, carcinoma, head and neck cancer, skin cancer, and nasopharyngeal cancer.

9. The method of claim 8, wherein the tumor is an esophageal cancer.

10. The method of claim 1, wherein the inflating memory CD8+ T cell response comprises production of CD8+/CX3CR1+/KLRG-1+T cells.

11. The method of claim 10, wherein the CD8+/CX3CR1+/KLRG-1+T cells maintain a memory effector phenotype for at least 30 days.

12. The method of claim 10, wherein the CD8+/CX3CR1+/KLRG-1+T cells form from 0.01% to 20% of total circulating CD8+ T cells in the subject.

13. The method of claim 10, wherein the CD8+/CX3CR1+/KLRG-1+T cells form from 2 percent to 20 percent of total circulating CD8+ T cells in the subject.

14. The method of claim 10, wherein the inflating memory CD8+ T cell response comprises production of CD8+/CX3CR1+/KLRG-1+T cells that have
(a) sustained expression of Tbx21,
(b) sustained expression of E2f2,
(c) low expression of Eomes
(d) low expression of PD-1,
(e) low expression of Tim-3, and/or
(f) low expression of Lag-3.

15. The method of claim 14, wherein the inflating memory CD8+ T cell response comprises production of CD8+/CX3CR1+/KLRG-1+/CD44+/CD62L−T cells.

16. The method of claim 15, wherein the inflating memory CD8+ T cell response comprises production of:
(a) CD8+/CX3CR1+/KLRG-1+/CD44+/CD62L−/CD27−/CD127−T cells,
(b) CD8+/CX3CR1+/KLRG-1+/CD44+/CD62L−/CD27(low)/CD127−T cells,
(c) CD8+/CX3CR1+/KLRG-1+/CD44+/CD62L−/CD27−/CD127(low) T cells, or
(d) CD8+/CX3CR1+/KLRG-1+/CD44+/CD62L−/CD27(low)/CD127(low) T cells.

17. The method of claim 1, wherein the adenoviral vector is replication-deficient and is human serotype 5 (AdHu5).

18. The method of claim 17, wherein the adenoviral vector lacks a sequence encoding a functional E1 protein and wherein the adenoviral vector lacks a sequence encoding a functional E3 protein.

19. The method of claim 1, wherein the adenoviral vector comprises:
(a) an upstream sequence that is upstream of the nucleotide sequence encoding the polypeptide comprising the single cancer-specific CD8+ T cell epitope, wherein the upstream sequence has at least 90% sequence identity to SEQ ID NO: 15: and
(b) a downstream sequence that is downstream of the nucleotide sequence encoding the polypeptide comprising the single cancer-specific CD8+ T cell epitope, wherein the downstream sequence has at least 90% sequence identity to SEQ ID NO: 33.

20. The method of claim 19, wherein the adenoviral vector further comprises:
(a) a sequence 5′ to the upstream sequence, wherein the sequence 5′ to the upstream sequence has at least 90% sequence identity to SEQ ID NO: 13; and
(b) a sequence 3′ to the downstream sequence, wherein the sequence 3′ to the downstream sequence has at least 90% sequence identity to SEQ ID NO: 14.

21. The method of claim 1, wherein the adenoviral vector comprises:
(a) a CMV promoter sequence as set forth in SEQ ID NO: 18:
(b) a translation initiation sequence as set forth in SEQ ID NO: 19; and
(c) a BGH polyadenylation sequence as set forth in SEQ ID NO: 34.

22. The method of claim 1, wherein tumor growth in the subject is controlled for 50 days or more after administration of the pharmaceutical composition.

23. The method of claim 1, wherein the single cancer-specific CD8+ T cell epitope is not a viral immunogen.

24. The method of claim 1, wherein the pharmaceutical composition is administered in combination with a therapy or a therapeutic agent.

25. The method of claim 24, wherein the therapeutic agent comprises an immune checkpoint inhibitor.

26. The method of claim 1, wherein the pharmaceutical composition is administered as a single dose.

27. The method of claim 1, wherein the pharmaceutical composition is administered as multiple doses.

28. A method of treating cancer in a subject in need thereof, comprising: administering a therapeutically effective amount of a pharmaceutical composition to a subject in need thereof, wherein the pharmaceutical composition comprises an adenoviral vector comprising a nucleotide sequence encoding a single cancer-specific CD8+ T cell epitope, and wherein the adenoviral vector does not comprise a nucleic acid molecule encoding a cancer-specific T cell epitope other than the single cancer-specific CD8+ T cell epitope.

29. An adenoviral vector comprising a nucleotide sequence encoding a single cancer-specific CD8+ T cell epitope, wherein the adenoviral vector does not comprise a nucleic acid molecule encoding a cancer-specific T cell epitope other than the single cancer-specific CD8+ T cell epitope, and wherein administration of the viral vector to a subject in need thereof induces an inflating memory CD8+ T cell response to the single cancer specific CD8+ T cell epitope in the subject.

30. A composition comprising: at least two adenoviral vectors, wherein each of the at least two adenoviral vectors comprises a nucleotide sequence encoding a polypeptide comprising a single cancer-specific CD8+ T cell epitope, wherein each of the at least two adenoviral vectors encodes a different single cancer-specific CD8+ T cell epitope, and wherein each of the at least two adenoviral vectors does not comprise a nucleic acid molecule encoding a cancer-specific T cell epitope other than the single cancer-specific CD8+ T cell epitope.


SEQUENCE LISTING (43 sequences)

SEQ ID NO: 1 - protein, 9 aa, Murine leukemia virus
SPSYVYHQF

SEQ ID NO: 2 - protein, 9 aa, Homo sapiens
SLLMWITQC

SEQ ID NO: 3 - protein, 11 aa, Murine leukemia virus
GGPESFYCASW

SEQ ID NO: 4 - protein, 27 aa, Mus musculus
VILPQAPSGPSYATYLQPAQAQMLTPP

SEQ ID NO: 5 - protein, 10 aa, Mus musculus
KYLSVQSQLF

SEQ ID NO: 6 - protein, 9 aa, Mus musculus
KYLSVQSQL

SEQ ID NO: 7 - protein, 9 aa, Human papillomavirus
RAHYNIVTF

SEQ ID NO: 8 - protein, 8 aa, Escherichia coli
ICPMYARV

SEQ ID NO: 9 - protein, 9 aa, Human papillomavirus
YPHFMPTNL

SEQ ID NO: 10 - protein, 9 aa, Human papillomavirus
HGIRNASFI

SEQ ID NO: 11 - protein, 8 aa, Human papillomavirus
SSPPMFRV

SEQ ID NO: 12 - protein, 8 aa, Human papillomavirus
TWYGFCLL

SEQ ID NO: 13 - DNA, 511 bp, Human papillomavirus
  source 1..511: mol_type = genomic DNA
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcgatc

SEQ ID NO: 14 - DNA, 30566 bp, Human papillomavirus
  source 1..30566: mol_type = genomic DNA
gatcgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 15 - DNA, 2191 bp, synthetic construct
  misc_feature 1..2191: note = Minigene immunogen cassette
  source 1..2191: mol_type = other DNA
acaagtttgtacaaaaaagcaggctccaccatgggaacccgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttggtacccccgccgccaccatg

SEQ ID NO: 16 - skipped

SEQ ID NO: 17 - DNA, 17 bp, synthetic construct
  misc_feature 1..17: note = attL1 sequence
  source 1..17: mol_type = other DNA
gtacaaaaaagcaggct

SEQ ID NO: 18 - DNA, 2100 bp, synthetic construct
  misc_feature 1..2100: note = CMV promotor
  source 1..2100: mol_type = other DNA
tgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgca

SEQ ID NO: 19 - DNA, 12 bp, synthetic construct
  misc_feature 1..12: note = Kozak sequence
  source 1..12: mol_type = other DNA
cccgccgccacc

SEQ ID NO: 20 - skipped

SEQ ID NO: 21 - DNA, 30 bp, Homo sapiens
  source 1..30: mol_type = genomic DNA
tctctgctgatgtggatcacccagtgctga

SEQ ID NO: 22 - DNA, 27 bp, Murine leukemia virus
  source 1..27: mol_type = genomic DNA
tctccatcttatgtgtaccaccagttc

SEQ ID NO: 23 - DNA, 33 bp, Murine leukemia virus
  source 1..33: mol_type = genomic DNA
ggaggccctgagtctttctactgtgcctcttgg

SEQ ID NO: 24 - DNA, 81 bp, Mus musculus
  source 1..81: mol_type = genomic DNA
gttatcttgcctcaagctcctagcggccccagctacgccacatatctgcaacctgctcaggcccagatgctgacccctcca

SEQ ID NO: 25 - DNA, 30 bp, Mus musculus
  source 1..30: mol_type = genomic DNA
aagtatctgtctgtgcagagccagctgttc

SEQ ID NO: 26 - DNA, 27 bp, Mus musculus
  source 1..27: mol_type = genomic DNA
aagtatctgtctgtgcagagccagctg

SEQ ID NO: 27 - DNA, 24 bp, Mus musculus
  source 1..24: mol_type = genomic DNA
atctgccctatgtacgccagagtg

SEQ ID NO: 28 - DNA, 27 bp, Mus musculus
  source 1..27: mol_type = genomic DNA
tatcctcacttcatgcccaccaacctc

SEQ ID NO: 29 - DNA, 27 bp, Mus musculus
  source 1..27: mol_type = genomic DNA
cacggcatcagaaacgccagcttcatc

SEQ ID NO: 30 - DNA, 24 bp, Mus musculus
  source 1..24: mol_type = genomic DNA
agcagccctcctatgttcagagtg

SEQ ID NO: 31 - DNA, 24 bp, Mus musculus
  source 1..24: mol_type = genomic DNA
acttggtacggcttttgcctgctg

SEQ ID NO: 32 - DNA, 27 bp, Mus musculus
  source 1..27: mol_type = genomic DNA
agagcccactacaacatcgtgaccttc

SEQ ID NO: 33 - DNA, 392 bp, synthetic construct
  misc_feature 1..392: note = Minigene immunogen cassette nucleotide sequence 3' to the T cell epitope sequence
  source 1..392: mol_type = other DNA
tgagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatcgatccgtcgagatatctagacccagctttcttgtacaaagtggt

SEQ ID NO: 34 - DNA, 225 bp, synthetic construct
  misc_feature 1..225: note = BGH poly A sequence
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 35 - DNA, 10 bp, synthetic construct
  misc_feature 1..10: note = attL2 sequence
  source 1..10: mol_type = other DNA
acccagcttt

SEQ ID NO: 36 - DNA, 15 bp, synthetic construct
  misc_feature 1..15: note = attR2 sequence
  source 1..15: mol_type = other DNA
cttgtacaaagtggt

SEQ ID NO: 37 - protein, 9 aa, Homo sapiens
SLLMWITQC

SEQ ID NO: 38 - DNA, 270 bp, synthetic construct
  misc_feature 1..270: note = AAV nucleotide sequence 5' to the minigene immunogen cassette
  source 1..270: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccg

SEQ ID NO: 39 - DNA, 130 bp, synthetic construct
  misc_feature 1..130: note = 5' ITR nucleotide sequence
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 40 - DNA, 140 bp, synthetic construct
  misc_feature 1..140: note = Extra sequences 5' to the minigene immunogen cassette
  source 1..140: mol_type = other DNA
tgtagttaatgattaacccgccatgctacttatctacgtagccatgctctaggaagatcggaattcgcccttaagctagctagttattaatagtaatcaattacggggtcattagttcatagcccatatatggagttccg

SEQ ID NO: 41 - DNA, 217 bp, synthetic construct
  misc_feature 1..217: note = AAV adenovirus nucleotide sequence 3' to the minigene immunogen cassette
  source 1..217: mol_type = other DNA
ctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 42 - DNA, 129 bp, synthetic construct
  misc_feature 1..129: note = 3' ITR nucleotide sequence
  source 1..129: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgca

SEQ ID NO: 43 - DNA, 87 bp, synthetic construct
  misc_feature 1..87: note = 5' Extra sequences 3' to the minigene immunogen cassette
  source 1..87: mol_type = other DNA
ctcgagttaagggcgaattcccgataaggatcttcctagagcatggctacgtagataagtagcatggcgggttaatcattaactaca